(19) United States
(12) Patent Application Publication
(10) Pub. No.: US 2026/0357029 A1
(43) Pub. Date: Nov. 12, 2026

(54) PET RECYCLING PROCESS

(71) Applicant: Imperial College Innovations Limited, London (GB)

(72) Inventors: Alain You LI, London (GB); Hui LUO, London (GB); Maria Magdalena TITIRICI, London (GB); Qilei SONG, London (GB); Dingchang YANG, London (GB)

(21) Appl. No.: 19/476,247

(22) PCT Filed: Apr. 18, 2024
(86) PCT No.: PCT/GB2024/051009

§ 371 Date: Oct. 17, 2025

(30) Foreign Application Priority Data
Apr. 19, 2023 (GB) 2305730.0

(51) Int. Cl. C08J 11/16; C07C 51/235; C07C 51/42; C25B 1/02; C25B 3/07; C25B 9/23; C25B 11/065; C25B 11/081; C25B 15/021

(52) U.S. Cl. CPC C08J 11/16; C07C 51/235; C07C 51/42; C25B 1/02; C25B 3/07; C25B 9/23; C25B 11/065; C25B 11/081; C25B 15/021; C08J 2367/03

(57) ABSTRACT

Described herein is a process for recycling polyesters such as polyethylene terephthalate (PET) into terephthalic acid (TPA) and ethylene glycol (EG) using mechanical or acoustic energy coupled with a low temperature aging process. Also described herein is a process for electrolytic conversion of ethylene glycol (EG) into clean hydrogen (H₂).

### FUNDING

The project leading to this application has received funding from the European Union's Horizon 2020 research and innovation programme under the Marie Sklodowska-Curie grant agreement No. 892614.

### TECHNICAL FIELD

The present disclosure is related to a process for degrading (such as recycling) polymeric plastic materials such as polyesters including polyethylene terephthalate (PET), and terephthalic acid (TPA) and ethylene glycol (EG) produced thereby.

### BACKGROUND OF THE INVENTION

Polyethylene terephthalate (PET) in the form of beverage bottles is the most recycled plastic. Yet, the majority of the PET produced annually currently still ends up in landfills or incineration facilities and, even worse, in our land and oceans.

Even for the small fractions that have been recycled, PET degrades through standard recycling processes (melting and reshaping) and, thus, recycled PET is normally unsuitable for producing food-grade containers due to leaching of degradation compounds.

Accordingly, there remains a need for improved processes to degrade and recycle polymeric plastic materials, such as PET materials.

### SUMMARY OF THE INVENTION

Described herein is a process for recycling polyesters such as polyethylene terephthalate (PET) into terephthalic acid (TPA) and ethylene glycol (EG), with subsequent electrolytic conversion of EG into clean H₂. Using mechanical or acoustic energy coupled with low temperature aging process, we are able to produce pure TPA that can be re-polymerised, e.g., to produce food packaging-grade PET. The EG electrolysis may enable production of H₂, but with a much lower thermodynamic energy requirement than water electrolysis. With a circular economy mindset, this process may enable the closing of the PET to TPA to PET usage loop, while at the same time contributing towards the decarbonisation of the H₂ production sector by bringing low-cost green H₂ to the market.

In a first aspect, the present invention provides a process for degrading PET (for example, recycling PET), comprising:
providing PET and mixing the PET with a base;
grinding the PET and base to obtain a ground mixture;
subjecting the ground mixture to aging conditions comprising about 75% or more relative humidity and about 30 °C or more for at least about 24 hours to form an aged ground mixture;
dissolving the aged ground mixture to form a solution comprising TPA, or a salt thereof, and EG;
extracting TPA from the solution to leave a solution comprising EG; and
electrolysing the solution comprising EG to produce hydrogen and glycolic acid.

The base may be any base suitable to hydrolyse the ester bonds in PET. The base may be an Alkali metal salt (for example, sodium hydroxide, lithium hydroxide or potassium hydroxide) or an Alkaline Earth metal salt (for example, calcium hydroxide or magnesium hydroxide), or a mixture thereof.

The PET and base may be mixed with a solvent. The solvent may be an aqueous solvent, optionally water. About 1 to about 5 weight equivalents of solvent may be used compared to PET, preferably about 1 to about 3 equivalents.

The grinding may comprise subjecting the mixture to a shearing force sufficient to form a ground mixture. The grinding may comprise mechanical degradation of a portion of the PET. The ground mixture may comprise PET, partially degraded PET, TPA, EG and/or unreacted base in varying proportions.

The grinding may comprise milling or acoustic mixing. The grinding may comprise milling, optionally wherein the milling is ball milling (e.g. vibratory ball milling or planetary ball milling).

Vibratory ball milling may be carried out at frequencies of greater than about 5 Hz, greater than about 10 Hz, greater than about 15 Hz, greater than about 20 Hz or greater than about 25 Hz, e.g. ranging from about 5-100 Hz, preferably between about 10-50 Hz, for example 30 Hz. Planetary ball milling may be carried out at rotational speeds of greater that about 100 rpm, greater than about 150 rpm, greater than about 200 rpm, or greater than about 250 rpm e.g. about 100 rpm to 600 rpm, preferably between about 200 rpm and 500 rpm, for example about 400 rpm.

The grinding may comprise milling or acoustic mixing, optionally using a resonance acoustic mixer (e.g. a Resodyn mixer such as the LabRAM II Resodyn acoustic mixer). Acoustic mixing may provide a mixing force of at least about 10 g (where g=9.81 m s⁻²) to the components, at least about 15 g, at least about 20 g, at least about 25 g, or at least about 30 g, as sufficient. Acoustic mixing may provide a mixing force of up to about 100 g.

The grinding may be carried out for at least about 1 minute, for at least about 2 minutes, for at least about 3 minutes, for at least about 4 minutes, for at least about 5 minutes or for at least about 10 minutes, optionally no more than about 30 minutes, about 25 minutes, about 20 minutes or about 15 minutes, for example for about 10 minutes, at any of the frequencies, mixing forces or rotational speeds described herein.

The amount of base may be equal to about 1 molar equivalent or more of the amount of PET, optionally about 1.5 molar equivalents or more, about 2 molar equivalents or more, for example about 3 molar equivalents. The amount of base may be equal to about 1 to about 3 molar equivalents of the amount of PET.

The aging conditions may comprise about 80% or more, about 85% or more, about 90% or more, about 95% or more, about 99% or more, or about 100% relative humidity, for example about 80 to about 100% relative humidity. The aging conditions may comprise temperatures of about 35 °C or more, about 36 °C or more, about 37 °C or more, about 38 °C or more, about 39 °C or more or about 40 °C or more, for example about 35 to about 45 °C. The aging may be carried out for at least about 24 hours, for at least about 30 hours for at least about 36 hours, for at least about 42 hours or for at least about 48 hours, optionally up to about 96 hours or up to about 72 hours, for example, about 24 to about 96 hours. Preferably, the ageing may be carried out for about 48 hours.

The aging may be carried out in the presence of water, for example in the presence of a salt solution (e.g. NaCl solution) or water.

The aged ground mixture may be dissolved in an aqueous solvent to form the solution. The solvent may be water.

Extracting TPA from the solution may leave a solution comprising a desired concentration of EG and base (e.g. NaOH). This solution may be used directly in the electrolysis. The desired concentration of EG and NaOH may therefore be such that it may be used directly in the electrolysis, for example, a concentration of 1 M of each of EG and NaOH.

Extracting terephthalic acid from the solution comprising TPA, or a salt thereof, and EG may comprise acidifying the solution, filtering and collecting the TPA residue. Extracting terephthalic acid from the solution comprising TPA, or a salt thereof, and EG may comprise electrodialysis. The electrodialysis may be carried out using a suitable membrane, for example ion exchange membranes.

The grinding and aging process described herein may be carried out in the absence (or with only a low amount) of solvent. This allows the solution to be prepared at a desired concentration (by adding a desired amount of solvent) enabling potential maximisation of TPA separation rate and minimum potential specific energy consumption during the electrodialysis process, as there is no excess solvent. Moreover, because no additional reagents are required to be added to the solution during electrodialysis separation of TPA, the solution containing desired concentration of EG and NaOH (without additional ions such as Cl⁻, CO₃²⁻, SO₄²⁻) can then be used for carrying out electrolysis process with high current density and glycolic acid selectivity.

The electrolysis may be carried out with anion exchange membrane (AEM). For example, the electrolysis may be carried out in a commercial membrane-electrode-assemble electrolyser with anion-exchange membrane (AEM), which resembling the water electrolyser and can easily scale up to higher H₂ production capacity (kg/hr). The solution comprising EG may be used as the anode electrolyte for electrolysis. The base may be sodium hydroxide (NaOH) and the process may further comprise adjusting the solution comprising EG to form an equimolar EG/NaOH solution, e.g. about 1 M EG/1M NaOH solution, prior to electrolysis. A NaOH solution may be used as the cathode electrolyte for electrolysis, optionally having an equal concentration with respect to NaOH as the anode electrolyte, for example about 1 M NaOH solution.

The electrolysis may be carried out at a temperature of at least about 40 °C, at least about 45 °C, at least about 50 °C, at least about 55 °C, or at least about 60 °C.

The anode for the electrolysis may be any suitable electrocatalyst material, for example, a Pt/C, Pd/C or Au/C electrode. The cathode for the electrolysis may be any suitable electrocatalyst material, for example, a Pt/C, Pd/C or Au/C electrode.

The electrolysis may comprise applying a current density of at least about 5 mA/cm² to the solution comprising EG, optionally applying a current density of at least about 10 mA/cm², at least about 15 mA/cm², at least about 20 mA/cm², at least about 25 mA/cm², at least about 30 mA/cm², at least about 35 mA/cm², at least about 40 mA/cm², at least about 45 mA/cm², at least about 50 mA/cm², at least about 75 mA/cm², optionally at least about 100 mA/cm². For example, about 5 to about 200 mA/cm².

The electrolysis may provide glycolic acid and hydrogen as product. The electrolysis may be carried out for a suitable amount of time to provide glycolic acid. The progress of the electrolysis can be quantified for example by measuring the hydrogen product, e.g. by gas chromatography.

The electrolysis may be carried out as a continuous process.

In a second aspect, provided herein is a process for degrading PET, comprising:
providing PET and mixing the PET with a base and an aqueous solvent, wherein 1 to 5 molar equivalents of each the base and solvent are used compared to the PET;
milling the PET mixture to obtain a ground mixture;
subjecting the ground mixture to aging conditions comprising about 75% or more relative humidity and about 30 °C or more for at least about 24 hours to form an aged ground mixture. The process may be used to form TPA.

The process may further dissolve the aged ground mixture to form a solution and extract TPA from the solution.

The base may be any base suitable to hydrolyse the ester bonds in PET. The base may be an Alkali metal salt (for example, sodium hydroxide, lithium hydroxide or potassium hydroxide) or an Alkaline Earth metal salt (for example, calcium hydroxide or magnesium hydroxide), or a mixture thereof.

The milling may comprise subjecting the mixture to a shearing force sufficient to form a ground mixture. The ground mixture may comprise PET, partially degraded PET, TPA, EG and/or unreacted base in varying proportions.

The milling may be ball milling (e.g. vibratory ball milling or planetary ball milling). Vibratory ball milling may be carried out at frequencies of greater than about 5 Hz, greater than about 10 Hz, greater than about 15 Hz, greater than about 20 Hz or greater than about 25 Hz, e.g. ranging from about 5-100 Hz, preferably between about 10-50 Hz, for example 30 Hz. Planetary ball milling may be carried out at rotational speeds of greater that about 100 rpm, greater than about 150 rpm, greater than about 200 rpm, or greater than about 250 rpm e.g. about 100 rpm to 600 rpm, preferably between about 200 rpm and 500 rpm, for example about 400 rpm. The grinding may be carried out for at least about 1 minute, for at least about 2 minutes, for at least about 3 minutes, for at least about 4 minutes, for at least about 5 minutes or for at least about 10 minutes, optionally no more than about 30 minutes, about 25 minutes, about 20 minutes or about 15 minutes, for example for about 10 minutes, at any of the frequencies or rotational speeds described herein.

The milling may be carried out by acoustic mixing, as described herein, for example optionally using a resonance acoustic mixer (e.g. a Resodyn mixer such as the LabRAM II Resodyn acoustic mixer). Acoustic mixing may provide a mixing force of at least about 10 g (where g=9.81 m s⁻²) to the components, at least about 15 g, at least about 20 g, at least about 25 g, or at least about 30 g, as sufficient. Acoustic mixing may provide a mixing force of up to about 100 g.

The aging conditions may comprise about 80% or more, about 85% or more, about 90% or more, about 95% or more, about 99% or more, or about 100% relative humidity, for example about 80 to about 100% relative humidity. The aging conditions may comprise temperatures of about 35 °C or more, about 36 °C or more, about 37 °C or more, about 38 °C or more, about 39 °C or more or about 40 °C or more, for example about 35 to about 45 °C. The aging may be carried out for at least about 24 hours, for at least about 30 hours for at least about 36 hours, for at least about 42 hours or for at least about 48 hours, optionally up to about 96 hours or up to about 72 hours, for example, about 24 to about 96 hours.

The aging may be carried out in the presence of water, for example in the presence of a salt solution (e.g. NaCl solution) or water.

Extracting TPA from the solution comprising TPA, or a salt thereof, and EG may comprise acidifying the solution, filtering and collecting the TPA residue. Extracting terephthalic acid from the solution comprising TPA, or a salt thereof, and EG may comprise electrodialysis. The electrodialysis may be carried out using a suitable membrane, for example ion exchange membranes.

In a third aspect, provided herein is a process for electrolysing EG, comprising:
providing a solution comprising EG and NaOH in water as anode electrolyte in an AEM electrolyser; and
applying a current density of at least about 5 mA/cm² to the solution to produce hydrogen and glycolic acid;
wherein the solution comprising EG and NaOH in water has a concentration of about 1 M EG and about 1M NaOH;
wherein the cathode electrolyte comprises an about 1 M NaOH solution;
wherein the anode for the electrolysis is a Pt/C electrode and/or where the cathode for the electrolysis is a Pt/C electrode; and
wherein the electrolysis process is carried out at a temperature of at least about 40 °C.

The electrolyser may be an AEM water electrolyser.

The electrolysis may be carried out at a temperature of at least about 45 °C, at least about 50 °C, at least about 55 °C, or at least about 60 °C.

The electrolysis may comprise applying a current density of at least about 10 mA/cm² to the solution comprising EG, optionally applying a current density of at least about 15 mA/cm², at least about 20 mA/cm², at least about 25 mA/cm², at least about 30 mA/cm², at least about 35 mA/cm², at least about 40 mA/cm², at least about 45 mA/cm², at least about 50 mA/cm², at least about 75 mA/cm², optionally at least about 100 mA/cm². For example, about 5 to about 200 mA/cm².

The anode for the electrolysis may be any suitable electrocatalyst material, for example, a Pt/C, Pd/C or Au/C electrode. The cathode for the electrolysis may be any suitable electrocatalyst material, for example, a Pt/C, Pd/C or Au/C electrode.

The electrolysis may provide glycolic acid and hydrogen as product. The electrolysis may be carried out for a suitable amount of time to provide glycolic acid. The progress of the electrolysis can be quantified for example by measuring the hydrogen product, e.g. by gas chromatography.

The electrolysis may be carried out as a continuous process.

In a fourth aspect, provided herein is TPA as prepared by a process according to the first or second aspect. The TPA may be high purity TPA.

In a fifth aspect, provided herein is a process or product as substantially herein described with reference to the accompanying figures.

Embodiments described herein in relation to the first aspect of the present invention apply mutatis mutandis to the second to fifth aspects of the present invention.

### SUMMARY OF FIGURES

FIG. 1 shows an illustration of the process described herein comprising in general three steps: i) mechanochemical depolymerisation, ii) TPA separation and iii) electrolysis of EG.

FIG. 2 shows (a) a flow chart of an exemplary electrodialysis process for extraction of TPA; (b) a schematic diagram of an exemplary electrodialysis processes; and (c) pictures of an exemplary experimental setup.

FIG. 3 shows a diagram of sampling treatment and measurement for NaOH recovery experiment.

FIG. 4 shows (a) pH variation of solution in concentrate chamber; (b) the variation of NaOH concentration in concentrate chamber; (c) NMR figure of finally obtained NaOH solution; (d) ethylene glycol concentration in dilute and concentrate chambers.

FIG. 5 shows (a) pH variation of solution in concentrate chamber; (b) the variation of NaOH concentration in concentrate chamber; (c) ethylene glycol concentration in dilute and concentrate chambers.

FIG. 6 shows a diagram of sampling treatment and measurement for TPA recovery.

FIG. 7 shows (a) the produced TPA and NaOH concentration as a function of time (mol/L), (b) The concentration of ethylene glycol in acid and salt chambers, respectively, (c) the distribution of ethylene glycol.

FIG. 8 shows (a) the produced TPA and NaOH concentration as a function of time (mol/L), (b) The concentration of ethylene glycol in acid and salt chambers, respectively, (c) the distribution of ethylene glycol.

FIG. 9 shows (a) specific energy consumption for NaOH recovery from depolymerized solution (kWh/mol); (b) specific energy consumption for TPA and NaOH production by BPM (kWh/mol).

FIG. 10 shows (a) specific energy consumption for NaOH recovery from depolymerized solution (kWh/mol); (b) specific energy consumption for TPA and NaOH production by BPM (kWh/mol).

FIG. 11 shows an image and component diagram of the AEM electrolyser system used for EG electrolysis.

FIG. 12 shows a. EG electrolysis reaction with Pt/C anode catalyst; b. the overall cell voltage recorded when holding the constant current density at 20, 40 and 100 mA cm⁻²; c. Faradaic efficiency of EG oxidation products and EG conversion rate at 10, 20, 40 and 100 mA cm⁻²; d. quantification of H₂ produced on the cathode side at 40 and 100 mA cm⁻².

FIG. 13 shows a. EG electrolysis reaction with Au/C anode catalyst; b. the overall cell voltage recorded when holding a constant current density at 20, 40 and 100 mA cm⁻²; c. Faradaic efficiency of EG oxidation products and EG conversion rate at 10, 20, 40 and 100 mA cm⁻².

### DETAILED DESCRIPTION

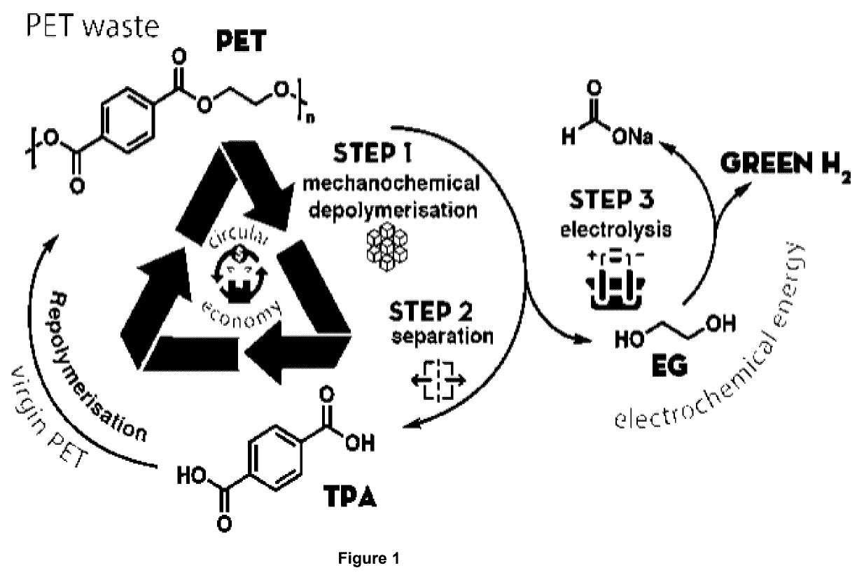

Described herein is a process for recycling polyesters such as polyethylene terephthalate (PET) into terephthalic acid (TPA) and ethylene glycol (EG), with subsequent electrolytic conversion EG into H₂ (FIG. 1). Using mechanical energy coupled with electrodialysis separation, we are able to produce pure TPA that can be re-polymerised, e.g., to produce food packaging-grade PET.

The additive impurities will not affect the purity of the TPA products, as the TPA remains as a solid fragment, which can be removed easily by filtration approach as part of the separation step of the process.

The EG electrolysis step enables production of H₂, but with a much lower thermodynamic energy requirement than water electrolysis. Substituting the challenging water oxidation (>1.23 V_{RHE}) with the partial oxidation of EG (<1 V_{RHE}) may save more than 50% electricity for H₂ production.

With a circular economy mindset, this described process enables closing the PET→TPA→PET usage loop, which will be extremely appealing for major food grade PET manufacturers, while at the same time contributing towards the decarbonisation of the H₂ production sector by bringing low-cost green H₂ to the market.

Described herein is a process for degrading PET, comprising:
providing PET and mixing the PET with a base;
   grinding the PET and base to obtain a ground mixture;
   subjecting the ground mixture to aging conditions comprising about 75% or more relative humidity and about 30 °C or more for at least about 24 hours to form an aged ground mixture;
   dissolving the aged ground mixture to form a solution comprising TPA, or a salt thereof, and EG;
   extracting TPA from the solution to leave a solution comprising EG; and electrolysing the solution comprising EG to produce hydrogen and glycolic acid.

PET is a polyester comprising repeating (C₁₀H₈O₄) units, for example of structure:

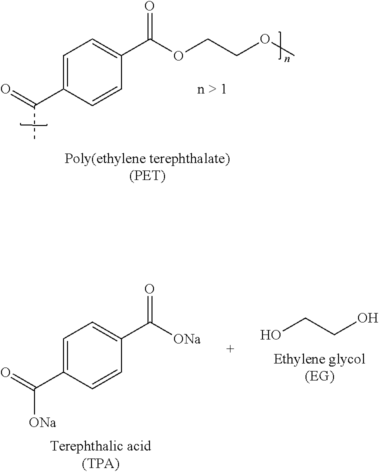

wherein n is an integer of greater than 1. PET polymers are formed from TPA and EG monomeric units.

The process described herein may be used to degrade PET. As used herein, the term “degrade” means chemically deteriorate, or depolymerise, the PET polymer material. Degrading, or depolymerising, the PET produces fragments of the polymer, monomers of the polymer, oligomers of the polymer, residues (or subunits) of monomers or any combination thereof. The degradation process described herein may comprise mechanical degradation (i.e. chemical degradation using mechanical energy input), chemical degradation and/or a combination thereof.

The PET degradation process described herein may produce TPA and EG monomers. These TPA and/or EG monomers may in turn be used in further downstream processing and, thus, the PET degradation process may be described as a recycling process for PET.

### Grinding the PET

Described herein is a process comprising providing PET and mixing the PET with a base and grinding the PET/base mixture to obtain a ground mixture. Thus, the process is a mechanochemical process for degrading PET.

Mechanochemical processes are in generally simple: two or more reactants are mixed and ground to promote chemical reaction using mechanical energy. The mechanical energy provided helps drive reactions by, for example, heating, reducing particle size, providing a new reaction interface, generating crystal defects and/or lattice displacement and/or melting of microdomains and/or polycrystalline phases and the like. Mechanochemical grinding may provide for mass transfer, similar to a “stirring” effect. These all contribute to the rapid reaction, with little need for solvents, e.g. in the solid phase.

Mechanochemistry may refer to the application of mechanical energy to condensed substances such as solids and liquids by means of shearing, friction, impact, extrusion and the like, inducing changes in their structure and physical and chemical properties, and inducing chemical reactions. Different from ordinary thermochemical reactions, mechanisation, the main power of the reaction is mechanical energy rather than thermal energy, so the reaction may be completed without harsh conditions such as high temperature and high pressure.

Strukil, ChemSusChem, 2021, 14, 330-338, the entire contents of which are incorporated herewith, described the mechanochemical depolymerisation of PET, using NaOH and ball-milling to obtain TPA and ethylene glycol (Scheme 1). The present invention builds on this work to degrade (i.e. depolymerise) PET.

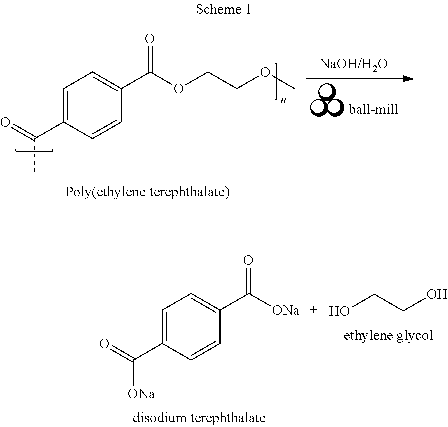

In the process described herein, the reaction mixture of PET and base is mixed using mechanical assistance (i.e. by grinding). The mechanical assistance may be in the form of milling, extrusion, (resonance) acoustic mixing, vortexing, or other methods of mixing solids, either intermittently, or continuously. Preferably, the grinding is carried out by milling or acoustic mixing. The grinding may be carried out by milling, for example by ball milling.

Ball-milling is an increasingly popular method to conduct chemical reactions, functional material preparation and waste treatment. The technique relies on using mechanical energy to induce chemical reactions (mechanochemistry). It requires minimal amounts to no solvents, allowing high reaction rates and can also circumvent substrate solubility issues. This is particularly interesting in the case of plastic upcycling, that are usually insoluble in common organic solvents.

A planetary ball mill may be employed in the milling, for example a TOB lab planetary ball milling machine. Alternatively, a vibratory mill was used, such as the Retsch MM400 mill used by Strukil. Both rely on the impact/shearing of one of more balls that may be of different sizes and materials (e.g. tungsten carbide, stainless steel, PTFE-covered steel, zirconium oxide, agate). Due to the shearing and impact forces on the reactants, a very efficient mixing of the materials can be achieved with minimal input of solvents.

Vibratory ball milling may be carried out at frequencies of greater than about 5 Hz, e.g. ranging from about 5-100 Hz, preferably between about 10-50 Hz, for example 30 Hz. Planetary ball milling may be carried out at rotational speeds of greater that about 100 rpm, e.g. about 100 rpm to 600 rpm, preferably between about 200 rpm and 500 rpm, for example about 400 rpm.

Alternatively, the grinding may be carried out with the assistance of flow ball milling. For example, Deasyl is a company that developed a flow ball-milling setup, using micro-beads as a milling medium in a cylindrical rotating chamber, described in WO 2019/228983A1, the entire contents of which are incorporated herein. Notably, their setup allows heating the chamber at up to 200 °C during grinding and has shown promising results for glycerol oligomerisation.

Alternatively, the grinding may be carried out with the assistance of acoustic mixing. For example, Resodyn acoustic mixers use sound energy at resonance frequency (58-62 Hz) and large displacement amplitude (2 cm) to mix powders at high energy (up to 100 g acceleration), this time without the requirements of milling balls. Friscid et al. Chemical Science, 2020, 11, 7578-7584, the entire contents of which are incorporated herein, showed the equivalence of this method compared to vibratory ball milling, with very similar if not better performances for organic transformations or MOF synthesis. According to the manufacturer, the technique can be enabled in flow and at the ton scale. An example of acoustic mixing is described in the examples using a LabRAM II Resodyn acoustic mixer.

Acoustic mixing utilizes low-frequency, high-intensity acoustic energy to achieve uniform mixing and grinding, potentially offering better homogeneity than traditional mixing methods. It can potentially significantly reduce processing times compared to conventional methods due to the efficient energy transfer and mixing mechanism and is potentially capable of processing a wide variety of materials, including liquids, powders, and pastes, and can handle different phases (solid-solid, liquid-liquid, solid-liquid) simultaneously. Acoustic mixing may also eliminate contamination from wear media, which is especially important in pharmaceuticals and high-purity applications. Acoustic mixing may also be more energy-efficient for certain applications, as the acoustic energy directly interacts with the material, reducing energy losses as it is kept at its resonant frequency to minimise energy losses during operation. Acoustic mixing processes may potentially be scaled from gram to 420 kg/batch scale with no loss in fidelity or re-optimisation of conditions needed.

In one embodiment, the grinding mixing (such as by ball milling) of the reaction mixture is for a duration of at least about 1 minute, for example from about 1 minute to about 1 hour. Preferably the mixing is for about 1 minute, about 2 minutes, about 3 minutes, about 4 minutes, about 5 minutes, about 6 minutes, about 7 minutes, about 8 minutes, about 9 minutes, or about 10 minutes.

The grinding may be carried out for sufficient time and at sufficient force to provide a ground mixture. A ground mixture may have an average particle size of less than about 5 mm.

Average particle size refers to the modal value of the particle size distribution of a sample. Particle size may be measured using laser diffraction and may be reported as volume equivalent sphere diameter.

The ground mixture may comprise PET, partially degraded PET and PET degradation products (i.e. TPA and EG) in varying proportions. The ground mixture may further comprise unreacted base and optionally a solvent.

The amount of base mixed with the PET may be equal to about 1 molar equivalent or more of the amount of PET, optionally about 1 molar equivalent or more, optionally about 3 molar equivalents.

The base may be any base suitable to hydrolyse the ester bonds in PET. The base may be an Alkali metal salt or an Alkaline Earth metal salt, or a mixture thereof. For example, the base may be an Alkali metal hydroxide or an Alkaline Earth metal hydroxide, or a mixture thereof, such as sodium hydroxide, lithium hydroxide, potassium hydroxide, calcium hydroxide or magnesium hydroxide, or a mixture thereof. The base is preferably sodium hydroxide.

The grinding may be carried out in the presence of a solvent. The solvent may be an aqueous solvent. The solvent may be water. Where the solvent comprises water, about 1 weight equivalent or more water to PET may be used.

Before grinding, the PET may be provided into small pieces, e.g. pieces each having a mean dimension of less than about 1 cm, less than about 9 mm, less than about 8 mm, less than about 7 mm, or less than about 6 mm, for example about 5 mm. A sample of small pieces of PET may have a distribution of pieces having mean dimensions of less than about 1 cm, for example about 0.1 mm to about 1 cm.

Material grinding is a well-established technique for physical grinding (for powder preparation for instance), but not for mechanochemical reactions i.e. to conduct chemical reactions using mechanical forces. Here we propose a more efficient solid-state route, with minimal energy input and alkali usage. This means there is an opportunity for a low-energy technique to emerge for PET recycling, with a plethora of grinding machines already available on the market.

Although the solo mechanochemical step has been previously reported, we have coupled it with mild condition aging process to make the whole system more efficient and energy-saving.

While we cannot exclude the grinding step (even over a short period of time) can induce partial degradation and depolymerisation, most of the depolymerisation occurs during the aging process.

### Aging the Ground Mixture

Described herein is a process comprising providing a ground mixture comprising PET and a base and optionally a solvent as described herein) and subjecting the ground mixture to aging conditions comprising about 75% or more relative humidity and about 30 °C or more for at least about 24 hours to form an aged ground mixture. The process described herein provides for the chemical degradation of PET.

Aging has low energy requirements, and we surmise that aging chambers consisting in low heating (e.g. less than about 50 °C) and humidity can be easily applied at industrial scale.

High relative humidity may be achieved by heating in the presence of water. For example, the mixture is heated to at least about 30 °C, for example, heated to temperatures of about about 35 °C or more, or about 40 °C or more.

The aging conditions comprise relative humidities of at least about 75%. For example, about 80% or more, about 85% or more, about 90% or more, about 95% or more, about 99% or more, or about 100% relative humidity.

The ageing may be carried out for at least about 36 hours, or for at least about 48 hours.

Both the grinding and aging processes described herein are easy to scale up, the static nature of aging means that mass transfer issues are minimised, along with somewhat easier heat transfer issues. This is all the truer for real-life PET samples of irregular size and hardness, that require extra energy for grinding.

### Extraction of Terephthalic Acid

Described herein is a process for extracting TPA from a solution comprising TPA, or a salt thereof, EG and a base, the process comprising dissolving a ground mixture comprising TPA, or a salt thereof, EG and a base to form a solution and extracting TPA from the solution to leave an EG solution.

The extraction of TPA, or a salt thereof, i.e. the separation of TPA, or salt thereof, and EG may be carried out by any suitable means known to the skilled person.

The ground mixture comprising TPA, or a salt thereof, EG and a base may be dissolved in an aqueous solvent. The solvent may be water.

The extraction may be carried out by acidifying the solution to precipitate TPA. For example, the solution may comprise a soluble salt of TPA and acidifying the solution forms TPA, which is insoluble in aqueous solvents. The insoluble TPA may then be extracted by, for example, filtration.

The solution may be acidified with any suitable inorganic acid, for example HCl or H₂SO₄ to achieve a pH of less than about 4, preferably less than about 3. The pH is preferably about 2 to about 3.

Alternatively, the extraction may be carried out by electrodialysis. Use of electrodialysis to extract TPA may be more economical and sustainable as it may be carried out without adding additional reagents and producing extra waste stream. For example, it is not necessary to add acid to the solution for electrodialysis, which thereby avoids neutralising the base in the solution. As a result, a particular advantage for electrodialysis is that the solution may be directly used in the electrolysis step as the base and EG are present, for example at the desired concentration, in the solution.

Electrodialysis may be used to separate the TPA, EG and the base. The base and EG may then be combined to form the solution for electrolysis.

The electrodialysis may be carried out using a suitable membrane, for example ion exchange membranes. Suitable membranes include anion exchange membranes, cation exchange membranes and bipolar membranes. More than one membrane may be used and combinations of membranes may also be used.

Described in the examples is method for electrodialysis to extract TPA from a solution comprising TPA, EG and a base. The method may be separated into two steps. In the first step, electrodialysis of the solution is carried out using a cation exchange membrane (e.g. a dialyser comprising 3 cation exchange membranes) to recover the base (e.g. NaOH). In the second step, electrodialysis is carried out using a bipolar membrane to separate TPA and EG.

Following this, the TPA may be precipitated.

The electrodialysis separation may comprise extraction of TPA and/or EG. The electrodialysis separation may be carried out as a multi-step process, for example, a two-step process. In the first step, one electrodialysis system separates the NaOH and Na₂TPA through an AEM membrane, with high purity EG solution obtained. In the second step, the NaOH/Na₂TPA solution is fed to another electrodialysis system with an ion-selective membrane that allow NaOH to transport while blocking the Na₂TPA. The NaOH can be concentrated through such electrodialysis system and reused for depolymerization, with high purity Na₂TPA solution obtained. The resulting Na₂TPA solution can be easily concentrated and converted to TPA by acid neutralization. In this process, as a large proportion of NaOH can be recycled from the solution, the amount of acid required for the neutralization would be significantly reduced, hence less salt waste would be generated.

The step of extracting TPA may preferably be carried out immediately after the step of ageing, i.e. within 60 minutes, 30 minutes, 20 minutes 15 minutes, 10 minutes, or 5 minutes.

The extracting step may remove substantially all the TPA, or salt thereof, from the solution to leave a solution substantially free of TPA, or salt thereof. For example, at least about 90% of the TPA, or salt thereof, may be extracted, preferably at least about 95% or at least about 99%, to leave a solution substantially free of TPA, or salt thereof, e.g. comprising less than about 10% by weight TPA or salt thereof, less than about 5% by weight or less than about 1% by weight.

The amount of TPA or salt thereof remaining in solution may be determined by spectroscopic techniques known to the skilled person, for example, NMR (¹H or ¹³C.

Also provided herein is TPA as prepared by a process described herein. The TPA obtained from the process described herein may be high purity TPA. For example, purity may be greater than about 90% (i.e. having less than about 10% by weight in impurities), greater than about 95%, greater than about 98%, greater than about 99%, or greater than about 99.9%.

The purity may be determined by spectroscopic techniques known to the skilled person, for example, NMR (¹H or ¹³C).

### Electrolysis of Ethylene Glycol

Described herein is a process for electrolysing a solution comprising EG and a base to produce hydrogen and glycolic acid.

Hydrogen is a critical enabler to transitioning to a zero-emission economy. The demand of hydrogen is on the rise: the forecast for the H₂ market is to exceed 200 Mt by 2028. However, as in today, hydrogen production is still primarily based on fossil fuels (out of 70 Mt annual hydrogen production globally, 76% is from natural gas and 23% from coal). This leads to 12-19 tCO₂/tH₂ emissions, representing 830 Mt of CO₂ per year. Green H₂ from electrolysis constitutes the most promising approach to reducing carbon footprint. However, the high cost due to significant electricity consumption remains a big challenge (the state-of-the-art water electrolyser have an electricity efficiency of 52 kWh/kg_{H2}, marking the green H₂ price at £4-5/kg); developing cost-effective routes for green hydrogen production is thus extremely timely.

On the other hand, the chemical sector is also among the largest industrial consumers of both oil and gas, accounting for 15% of total primary demand for oil on a volumetric basis and 9% of gas. The use of fossil-fuels as feedstock and energy source, also dictates its unsustainability with direct CO₂ emission of 880 Mt in 2018. The “UN 2030 Agenda for Sustainable Development” stresses the importance of the fuel and chemical industry to be low-carbon, resource-efficient, creating a circular economy. While the demand for fuels and chemicals is projected to grow in parallel with economic activity, to get on track in the transition towards net-zero emission, efforts are needed to address the CO₂ emissions from all stages in the value chains.

Electrochemical conversion of PET waste derived EG solution, separated from the PET depolymerisation process (step 1 & 2) could potentially offer a direct and cost-effective route for the sustainable transition of hydrogen and chemical industry, by generating hydrogen and valuable chemicals simultaneously. The EG electrolysis step resembles water electrolysis, with a much lower thermodynamic energy requirement. Substituting the challenging water oxidation (>1.23 V_{RHE}) with the partial oxidation of EG (<1 V_{RHE}) saves more than 50% electricity for H₂ production. Our targeted chemical is glycolic acid, the simplest α-hydroxy acid that can be used in skin care products, chemical cleaning, biodegradable materials and the pharmaceutical industry. The GA market was 468.2 million USD in 2020 and was projected to reach 820.3 million USD by 2027, at a potential compound annual growth rate of 7.3%.

In the process described herein, the reactions happening on the anode and cathode may be as follows:
Anode: C₂H₆O₂+4OH⁻→3H₂O+4e⁻+C₂H₄O₃
Cathode: 2H₂O+4e⁻→4OH⁻+2H₂
Overall reaction: C₂H₆O₂+H₂O→C₂H₄O₃+2H₂

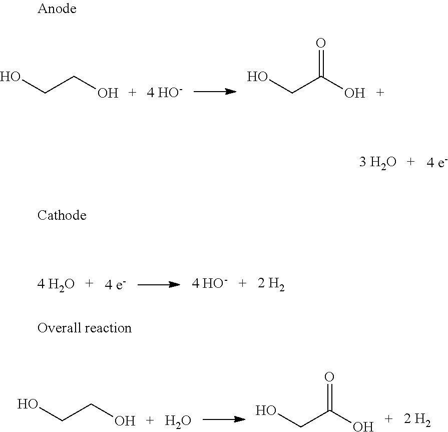

The electrolysis may be carried out in an anion exchange membrane (AEM) electrolyser. For example, an AEM water electrolyser may be used.

The base for the electrolysis may be the same or different from the base for the PET degradation (grinding and ageing steps). Preferably, the base is the same throughout the process. In the electrolysis, the base provides alkaline conditions for the electrolyte solution. The base may be, for example, sodium hydroxide (NaOH).

The solvent for the electrolysis (i.e. used to form the solution comprising EG and a base) may be water.

The solution comprising ethylene glycol may be used as the anode electrolyte for electrolysis. The process may further comprise adjusting the solution comprising ethylene glycol to form an about 1 M EG/1 M NaOH solution prior to electrolysis. For the cathode electrolyte, a NaOH solution may be used, optionally an about 1 M NaOH solution.

The electrode may comprise an electrocatalyst material. The anode and/or cathode may be any suitable materials for a water electrolysis, such as Pt/C, Pd/C or Au/C. For example, the anode may be a Pt/C electrode and/or the cathode may be a Pt/C electrode.

The electrolysis may comprise applying a current density of at least about 5 mA/cm² to the solution comprising EG. For example, electrolysis may comprise applying a current density of at least about 10 mA/cm², at least about 20 mA/cm², optionally at least about 30 mA/cm², optionally at least about 40 mA/cm², optionally at least about 50 mA/cm², optionally at least about 75 mA/cm², optionally at least about 100 mA/cm².

The electrolysis may be carried out at a temperature of at least about 40 °C, optionally at least about 50 °C, optionally at least about 60 °C.

Electrolysis may be carried out for at least about 5 minutes, for at least about 10 minutes, at least about 20 minutes, at least about 30 minutes or at least about 60 minutes.

Accordingly, also described herein is a process for electrolysing EG, comprising:
providing a solution comprising EG and NaOH in water as anode electrolyte in an AEM electrolyser; and
applying a current density of at least about 5 mA/cm² to the solution to produce hydrogen and glycolic acid;
wherein the solution comprising EG and NaOH in water has a concentration of about 1 M EG and about 1M NaOH;
wherein the cathode electrolyte comprises an about 1 M NaOH solution;
wherein the anode for the electrolysis is a Pt/C electrode and/or where the cathode for the electrolysis is a Pt/C electrode; and
wherein the electrolysis process is carried out at a temperature of at least about 40 °C.

The electrolysis process may be carried out as a continuous process.

The high glycolic acid selectivity (e.g. greater than about 90%) is among the best performance reported so far. The process described herein enables high rate of production of glycolic acid, e.g. at about 50 mA cm⁻² or greater, about 75 mA cm⁻² or greater or 100 mA cm⁻² or greater.

Based on the theoretical mass balance, for each ton of PET converted, 36 kg green H₂, 747 kg TPA and 212 kg glycolic acid can be produced, which may create a net added value of $1002/ton PET under current market price, which once scaled up can create significant economic value.

The advantages of the present invention include, but are not limited, to the following:
It allows the usage of end-of-life PET wastes, rather than the non-degraded bottles that are ready to be re-processed
It does not involve high temperature, high pressure treatment, and the whole process can be entirely electrified.
The depolymerisation step is a solid-state process, no solvents are needed.
It produces high purity monomers for food-grade packaging
It generates hydrogen and glycolic acid on the side, providing additional revenue streams.

Throughout the description and claims of this specification, the words “comprise” and “contain” and variations of the words, for example “comprising” and “comprises”, mean “including but not limited to”, and are not intended to (and do not) exclude other components. In any of the embodiment described herein, reference to “comprising” also encompasses “consisting essentially of”.

Features described above in relation to each aspect of the present invention also represent features of each other aspect of the present invention subject to a technical incompatibility that would prevent such a combination of preferred features. Furthermore, it will be evident to the skilled person that advantages set out above in respect of each aspect of the present invention are also offered by each other aspect of the present invention.

### EXAMPLES

The following examples are merely illustrative examples of the invention described herein and are not intended to be limiting upon the scope of the invention.

### Example 1—Mechanochemical PET Degradation and Separation of TPA

Sodium hydroxide (reagent grade, >98%, pellets (anhydrous)) and dimethyl sulfone (qNMR) were purchased from Sigma-Aldrich without further purification. Planetary ball-mill used was purchased from Xiamen Tob New Energy Technology Co., Ltd. (TOB-DSP-LBPBM06A-0.4L). NMR spectra were recorded with a Jeol (¹H, ¹³C, 400 MHz) spectrometer. PET was obtained from Starbucks cold drink cups, Buxton and Highland Spring water bottles.

Ball milling. In a typical procedure, PET pieces were cut in tiny square pieces (5×5 mm). 1 g of PET was loaded into the milling jar (50 mL, ZrO₂), followed by NaOH and deionised water (3 molar equivalents each, assuming a molecular weight of 192 g/mol for the PET repeat unit). 8 ZrO₂ balls were added (10 mm diameter, 26 g) total, and the milling jar was capped and wrapped in parafilm. The ball-milling parameters were as follow: 5 mins of grinding clockwise at 400 rpm, followed by 2 mins of rest, then 5 mins of grinding in the opposite direction at the same speed. After opening the jar, the mixture had the appearance of PET pieces of the same size than before, covered with a fine layer of NaOH, that were easy to scrape out from the jar. The balls were removed, and the remaining solids were collected in a 5 mL glass vial.

Aging. The glass vial was then transferred into a 150 mL screw cap glass bottle, layered with c.a. 5 mm of deionised water at the bottom, to ensure a 100% relative humidity (RH) during aging. For some experiments, 75% RH was attained using a saturated NaCl aqueous solution. Up to 4 samples can be aged at a time with this setup. The bottle was then capped, sealed with parafilm, and transferred to an oven programmed at 45 °C for 48 h.

Work-up. After aging, the pieces became more brittle and became darker in colour. The glass vial was taken out of the bottle, to which 5 mL distilled water was added and the vial was taken to sonicate for 30 s. The water was transferred to a beaker, and the operation was repeated 3 times to ensure all the pieces were transferred into the beaker. DI water was added so that the total volume amounted to 30 mL. The solution was then vacuum-filtered and rinsed with another 10 mL twice. The filtrate was transferred to a 250 mL round-bottom flask before analysis.

TPA separation. To separate the TPA, a few drops of concentrated HCl can be added to the solution until pH turns to 1. TPA can then easily be filtered out as a white powder, and analysed by NMR in deuterated DMSO instead of D₂O.

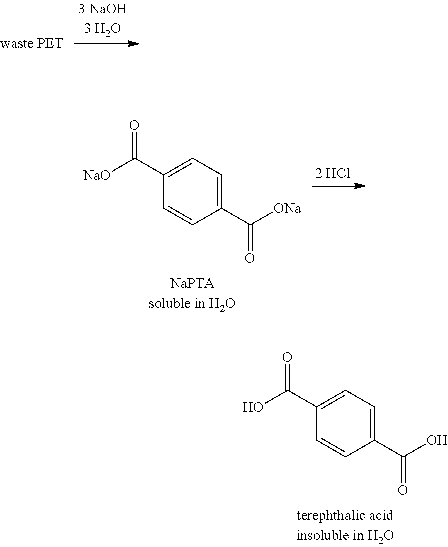

Analysis. To the solution flask was added a known amount of dimethyl sulfone NMR standard (between 10-15 mg). The mixture was swirled, then a 0.5-1 mL aliquot was transferred to an NMR tube, to which was added 100 μL of D₂O. The program used for NMR analysis is named “solvent suppression”, with D₂O as the solvent.

The yields were calculated as follow:

$$Yield ( \% ) = ( ( \frac{{m}_{S t d}}{{M}_{S t d}} ) \times ( \frac{{Int}_{P r o d}}{{Int}_{S t d}} ) \times ( \frac{{N}_{S t d}}{{N}_{P r o d}} ) \div ( \frac{m P E T}{192} )$$

With m_{Std} is the amount of NMR standard used (g), M_{Std} the molecular weight of the standard (g/mol, 94 for dimethyl sulfone), Int_{Prod} and Int_{Std} the NMR area integration under the peak of the product and the standard respectively, N_{Std} and N_{Prod} the proton count of the standard and the product (N_{dimethyl sulfone}=6, N_{TPA}=4, N_{EG}=4), m_{PET} is the amount of PET (g) introduced.

NMR was used throughout the study to identify and quantify the soluble products produced after milling and aging. In a typical procedure, three singlet peaks with very distinct chemical shifts were observed in the mixture: disodium terephthalate (4H, s, 7.76 ppm), ethylene glycol (4H, s, 3.55 ppm) and dimethyl sulfone (6H, s, 3.03 ppm). Very minor peaks stemming remaining EG/TPA oligomers can be seen but they were negligible (<0.1 mol % relative to TPA and EG)

After acidification and filtering, re-protonated TPA can be easily filtered, and analysed in DMSO, showing high purity of the recovered TPA. ¹H NMR (400 MHz, [D₆]DMSO, 25 °C, TMS): δ=8.04 ppm (s, 4H; Ar—H). ¹³C NMR (400 MHz, [D₆]DMSO, 25 °C, TMS): δ=167.2 (COOH), 135.0 (C), 130.0 ppm (CH).

### Results and Discussions

TABLE 1
Direct ball milling of PET optimisation. (note: except for entries 1-2, the amount of
PET was changed to accommodate the addition of solid additives, to stick to roughly 1/10
weight ratio between the solids and the balls weight)

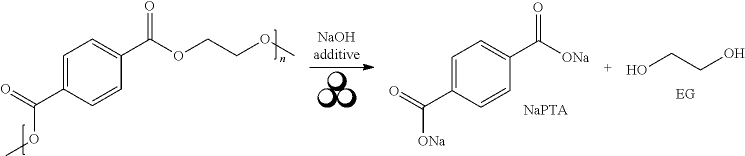

| | | | Milling | Yield (EG/Na-TPA |
|---|---|---|---|---|
| Entry | PET (g) | Chemicals | Time | %) |
| 1 | 3 | 1 eq NaOH/H₂O | 4 h | 0.61/0.45 |
| 2 | 1 | | 6 h | 8.8/12.3 |
| 3 | 0.3 | 1 eq | 6 h | 4.8/3.2 |
| | | NaOH/H₂O/Al₂O₃ | | |
| 4 | 1 | 1 eq NaOH/MeOH | 6 h | 2/3.1 |
| 5 | 0.5 | 1 eq NaOH/CaO | 6 h | 1.5/1.2 |
| 6 | 1 | 3 eq NaOH/H₂O | 6 h | 38/26 |
| 7 | | | 6 h^{[a]} | 64/95 |

26 g of ZrO₂ balls (10 mm, 8 total) were used except for entry 1 (30 g of 2 mm ZrO₂ balls). Milling was performed at 400 rpm, alternating between both directions with 10 mins cycles each, with a 5 mins rest cycle in between.
^{[a]}Aging at 45 °C, 76% RH for 3 days was performed after milling.

Initial conditions were conducted with 3 g of PET sample, with 1 equivalent of NaOH and water to depolymerise it. After 4 h of milling less than 1% yield was observed for both EG and Na-TPA (Table, entry 1). Decreasing the PET amount in the milling jar while increasing the milling time from 4 h to 6 h increased the EG/Na-TPA yield to 8.8/12.3% (Table, entry 2). Using basic grinding additives, such as Al₂O₃ and CaO, or an organic solvent such as MeOH (Table, entries 3-5) drastically decreased the yield to less than 5% for each component. Increasing the excess of NaOH/H₂O to 3 equivalents allowed to obtain a 38/26% EG/Na-TPA yield (Table, entry 6). We tried aging the sample after milling at 45 °C, 75% relative humidity for 3 days, which massively increased the yield to 64/95% (Table, entry 7).

TABLE 2
Ball-milling of PET followed by aging at 75% RH.

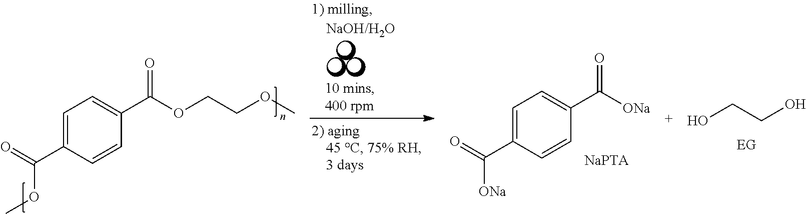

| Entry | NaOH (eq.) | H₂O (eq.) | Yield (EG/Na-TPA %) |
|---|---|---|---|
| 1 | 3 | 3 | 64/95 |
| 2 | | | 71/100 |
| 3 | 5 | 5 | 73/100 |

26 g of ZrO₂ balls (10 mm, 8 total) were used. Milling was performed at 400 rpm, with the following cycles: 5 mins forward, 2 mins rest then 5 min backwards. Aging was performed at 45 °C, 75% RH

Encouraged by the effect of aging, we tested shorter milling times (10 mins) followed by 3 days aging, increasing the EG/Na-TPA yield to 64/95-71/100%, with an excellent reproducibility (Table, entries 1-2). Increasing the NaOH/H₂O excess did not provide a significant performance boost, with a 73-100% yield (Table, entry 3).

TABLE 3
Ball-milling of PET followed by aging at 100% RH.

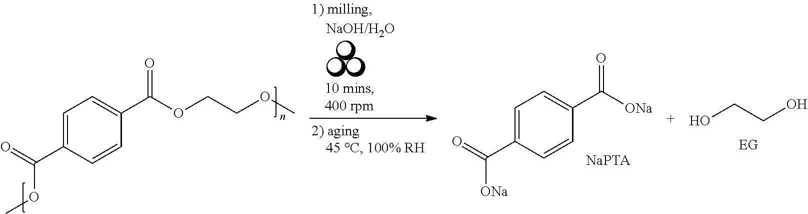

| Entry | NaOH (eq.) | H₂O (eq.) | Aging conditions | Yield (EG/Na-TPA %) |
|---|---|---|---|---|
| 1 | 2 | 2 | 3 days | 38/60 |
| 2 | | | | 62/97 |
| 3 | 3 | 3 | 2 days | 75/100 |
| 4 | | | | 72/99 |
| 5 | 3 | 3 | 1 days | 33/51 |

26 g of ZrO balls (10 mm, 8 total) were used. Milling was performed at 400 rpm, with the following cycles: 5 mins forward, 2 mins rest then 5 min backwards. Aging was performed at 45 °C, 100% RH.

We then sought to optimise the reaction conditions by introducing a lower amount of NaOH/H₂O, which not only reduced the yield but gave a lower reproducibility 38/60-62/97% (Table, entries 1-2). Decreasing the aging time to 2 days while sticking to 3 equivalents of NaOH/H₂O preserved both yield and reproducibility 75/100-72/99% (Table, entries 3-4). Further decreasing the aging time however drastically affected the performance (33/51% yield, Table, entry 5). 3 equivalents of NaOH/H₂O gave the best performance, and the solid mixture after aging consisted in PET pieces evenly covered with NaOH.

### Conclusion

3 equivalents of NaOH/H₂O, with 10 mins of milling and 2 days of aging and afforded 7500 EG and 1000% Na-TPA.

Here we laid the groundwork for further scalability of mechanochemical depolymerisation of PET, by transposing the seminal work of Štrukil to planetary ball-milling. For further scaling up, we have identified two other milling devices with very few applications in the scientific literature, compared to vibratory ball-milling.

Here we propose a more efficient solid-state route, with minimal energy input and alkali usage. Ball-milling is still an underexplored method, and this is even more the case for aging technique despite its operational simplicity. Material grinding is a well-established technique for physical grinding (for powder preparation for instance), but not for mechanochemical reactions i.e. to conduct chemical reactions using mechanical forces. This means there is an opportunity for a low-energy technique to emerge for PET recycling, with a plethora of grinding machines already available on the market

Aging has low energy requirements, and we surmise that aging chambers consisting in low heating (<50 °C) and humidity can be easily applied at industrial scale.

### Example 2—Resonance Acoustic Mixing for PET Depolymerisation

Resodyn Acoustic Mixing technology utilises low-frequency, high-intensity acoustic energy to mix materials uniformly and efficiently. Unlike ball milling that relies on mechanical forces generated by milling media, Resodyn Acoustic Mixers create a three-dimensional mixing action that can process a wide variety of materials, including solids, liquids, powders, pastes, and slurries.

In this example, a LabRAM II Resodyn acoustic mixer was used to depolymerise PET plastic into its monomers terephthalic acid (TPA) and ethylene glycol (EG).

Materials: All materials were purchased from Sigma Aldrich and were used as received with the exception of the PET plastic which was obtained from a waste management company as a “real-world” sample. Deionised water was also used in the process and obtained from Imperial College London.

Methods and results: PET waste plastic was weighed out into vials and then NaOH was added as the catalyst with deionised water. The vials were sealed with caps and then placed into the vial holders and onto the LabRAM II mixer stage. Mixing parameters of 1 g-20 g scale with various stoichiometric ratios of base and H₂O as well as screening the oscillating force at 30 g, 60 g and 90 g was done.

The samples were then removed from the Resodyn mixer and aged thermally at 45 °C for 48 hours under low energy conditions. Samples were then taken out of the oven, cooled to room temperature and deionised water was added to the samples and they were ultrasonicated in a water bath for 10 minutes. The samples were then vacuum filtered to remove plastic additives and give the mixture of disodium terephthalate (Na-TPA) and EG.

Subsequently, quantitative NMR (q-NMR) was performed on the filtrate to calculate the yields of Na-TPA and EG. Using dimethyl sulfone as the reference. For a 20 g batch of PET the Resodyn acoustic mixing method (15 min, 90 g, 48 hours at 45 °C in the oven) gave the largest yields of EG and Na-TPA (78% and 100% yield respectively) which already surpass the previous ball milling methods.

### Conclusions:

In conclusion we have showed that mechanochemical depolymerisation by Resodyn acoustic mixing with the LabRAM II mixer is an effective way to depolymerise waste PET plastic into its monomers. The results show an improved depolymerisation yield of 78% (EG) and 100% (TPA) compared to previous ball milling experiments

### Example 3—TPA Extraction by Electrodialysis

In this example, we report a membrane based electrochemical process-electrodialysis (ED) for effective separation and recovery of NaOH and TPA. The whole process is separated into two steps.

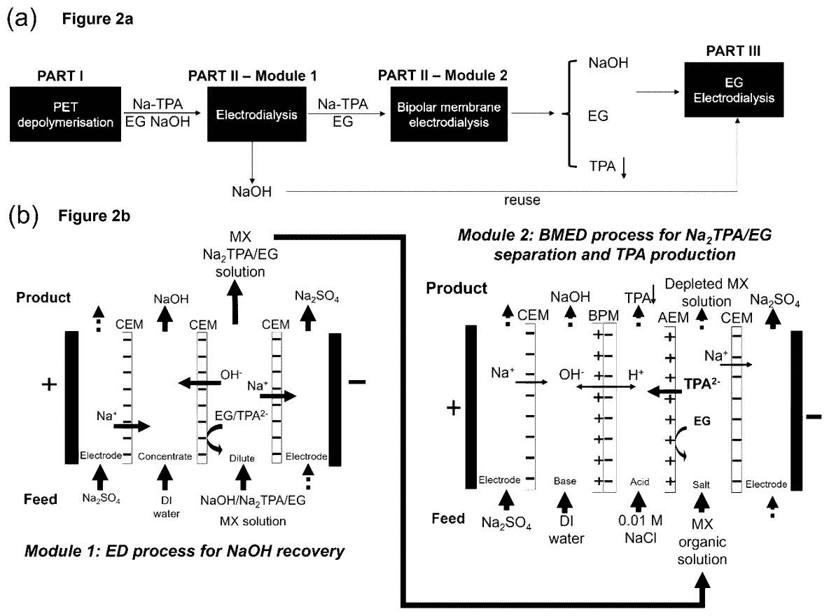

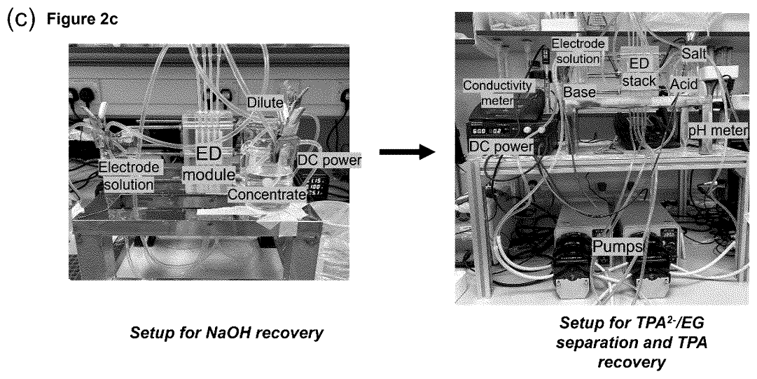

In the first step, electrodialysis system equipped with three cation exchange membranes for NaOH recovery. In the second step, electrodialysis system equipped with bipolar membrane (BPM) for Na₂TPA and EG separation and TPA precipitation (as shown in FIG. 2(a)). As shown in FIG. 2(b), for NaOH recovery step, this invention uses a lab-scale ED stack equipped with three cation exchange membranes (CEMs, Nafion), placed in parallel. The effective area of each membrane was 20 cm², and they were separated by a 1 cm-thick channel. The ED stack is operated in a galvanostatic mode controlled by DC power. The voltage is recorded every half an hour. The pH of concentrate solutions is recorded every half an hour. The mixed NaOH/Na₂TPA/EG solution and Na₂SO₄ solution are circulated through the dilute and electrode chambers of the ED system, and the DI water is circulated through concentrate chambers of the ED system, respectively, by peristaltic pumps. For bipolar membrane electrodialysis (BMED) process, this invention uses a lab-scale BMED stack equipped with only one triplet of anion-exchange membranes, cation exchange membrane, and bipolar membrane placed in parallel. The effective area of each membrane is 20 cm². 1 cm-thick channel separated two neighbouring membranes. The BMED stack is operated in a galvanostatic mode controlled by DC power. The voltage is recorded every half an hour. The pH of acid and base solutions is recorded every half an hour. The mixed Na₂TPA/EG solution and Na₂SO₄ solution are circulated through the salt and electrode chambers of the BMED system. The DI water and 0.01 M salt solution are circulated through the acid and base chambers of the BMED system, respectively, by peristaltic pumps. The electrode solution was pumped through both anode and cathode chambers. The pictures of the ED and BMED setup are shown in FIG. 2(c).

### Electrodialysis (ED) for NaOH Recovery

### Material and Method

### Chemicals and Membranes

Cation exchange membrane (CEM, denoted as Nafion) was used in this study. Membranes' characteristics are listed in Table 4. All the chemicals used in this study were of analytical grade, and all the solutions were prepared using Milli-Q water throughout the study.

TABLE 4
Characteristics of commercial CEM used in this study.

| | | Ion | | | |
|---|---|---|---|---|---|
| | Thick- | exchange | Ion | Water | Pre- |
| Mem- | ness | capacity | conductivity | uptake | treatment |
| brane | (μm) | (meq./g) | (mS/cm) | (wt %) | method |
| CEM | 50 | 0.93 | 145 | 35 | 0.1M NaCl, |
| | | | | | 12 hrs |

### Experimental Design

In the ED experiments, the mixed NaOH/Na₂TPA/EG solution and DI water were pumped into dilute and concentrate chambers separately, with a volumetric flow rate of 60 mL/min. Na₂SO₄ was pumped into electrode chamber, with volumetric flow rate of 120 mL/min. The ED experiments were performed in a batch mode in which the effluent streams were circulated back to the respective tanks. The experiment took 4 days in total. Samples were collected every 12 hours to measure the concentration of OH⁻ by using titration method.

The total experiment was separated into two batches. The first batch lasted two days with applied current density 15 mA/cm². After two days, NaOH product circulated in the concentrate chamber was replaced by DI water with a volume of 200 mL and started the second batch, which was also operated for two days with applied current density 15 mA/cm².

### Sample Treatment and Testing Methods.

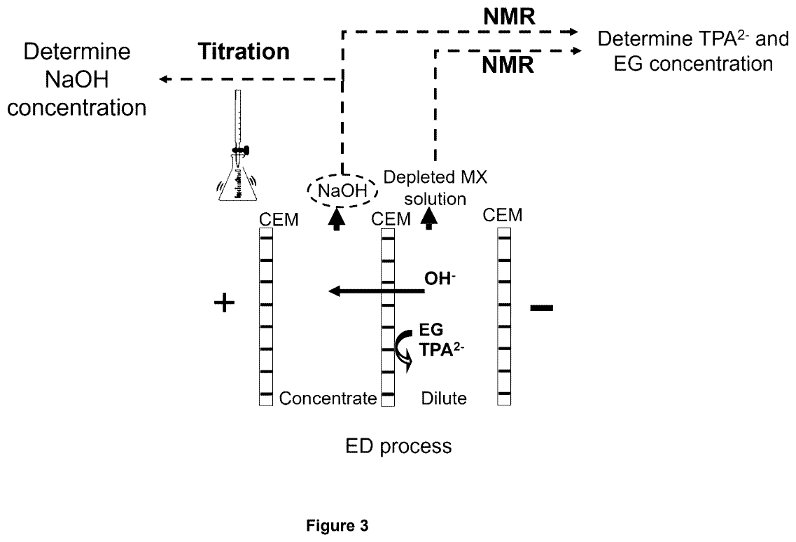

As seen from FIG. 3, to have a more precise determination of NaOH concentration of the recovered NaOH solution from the concentrate chamber, 1.0 M H₂SO₄ solution was used for titration. The sample taken from concentrate and dilute chamber was also used for detecting the concentration of ethylene glycol and TPA. d6-DMSO is used as the NMR solvent. The concentration of TPA is calculated by integrating the NMR proton peaks relative to an internal standard (dimethyl sulfone) of a known concentration.

### Electrodialysis Process for Simulated Depolymerized Solution Treatment

TABLE 5
Initial composition of feed solutions

| | | Concentration | Volume |
|---|---|---|---|
| Solution | Chemical | (mol/L) | (mL) |
| Concentrate | NaOH | 0.6 | 400 |
| | EG | 0.59 | |
| | Na₂TPA | 0.59 | |
| Dilute | DI water | NA | 200 |
| Electrode | Na₂SO₄ | 0.3 | 400 |

### Results

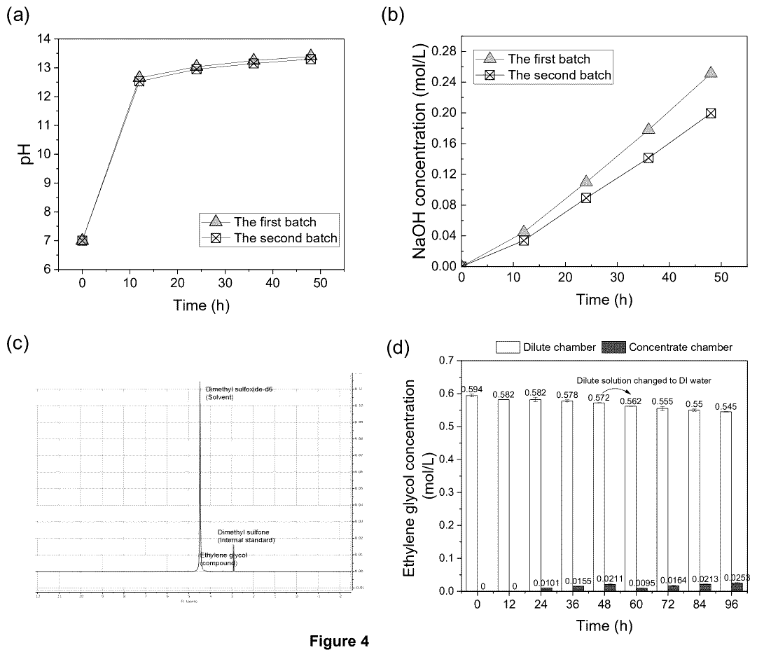

Acid and base pH as a function of time is shown in FIG. 4(a). As can be seen from the figure, the pH of concentrate solution could increase from around 7 to around 13.4 during the first batch, and the pH could reach 13.3 during the second batch. Therefore, about 0.25 M NaOH was recovered from mixed organic solution in the first batch and around 0.2 M NaOH was recovered in the second batch (FIG. 4(b)). The NaOH recovery efficiency in the second batch is lower than that in the second batch, and may be due to the lower initial NaOH concentration for the second batch.

To further explore the purity of obtained NaOH and the loss of ethylene glycol and TPA²⁻, samples from NaOH product were measured by NMR and HPLC. From FIG. 4(c) which shows the NMR figure of the final obtained NaOH solution, there was no presence of TPA²⁻ ion, meaning that the Nafion membrane could successfully separate OH⁻ and TPA²⁻. From the HPLC result, around 0.02 M ethylene glycol leaked from the feed solution to NaOH solution in the first batch and 0.025 M in the second batch (FIG. 4(d)).

### Related Electrodialysis Processes for Real Depolymerized Solution Treatment

TABLE 6
Initial composition of feed solutions

| | | Concentration | Volume |
|---|---|---|---|
| Solution | Chemical | (mol/L) | (mL) |
| Concentrate | NaOH | 1.4 | 200 |
| | EG | 0.29 | |
| | Na₂TPA | 0.3 | |
| Dilute | DI water | NA | 200 |
| Electrode | Na₂SO₄ | 0.3 | 400 |

### Results

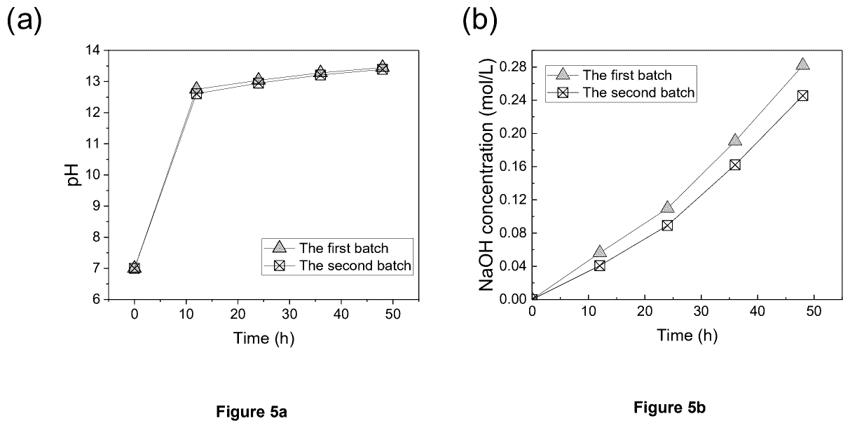

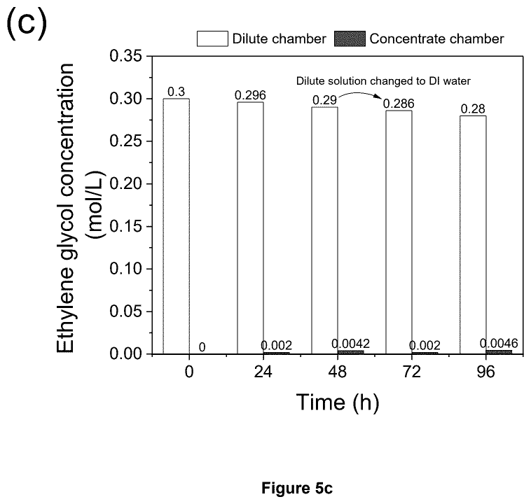

Acid and base pH as a function of time is shown in FIG. 5(a). As can be seen from the figure, the pH of concentrate solution could increase from around 7 to around 13.45 during the first batch, and the pH could reach 13.4 during the second batch. Therefore, about 0.28 M NaOH was recovered from mixed organic solution in the first batch and around 0.25 M NaOH was recovered in the second batch (FIG. 5(b)).

To further explore the purity of obtained NaOH and the loss of ethylene glycol and TPA²⁻, samples from NaOH product were measured by HPLC and NMR. From the HPLC result, there was around 0.0042 M ethylene glycol leaked from feed solution to NaOH solution in the first batch and 0.0046 M in the second batch (FIG. 5(c)).

### Bipolar Membrane Electrodialysis (BMED) for Disodium Terephthalate (Na₂TPA) and Ethylene Glycol (EG) Separation and Terephthalic Acid (TPA) Conversion

### Material and Method

### Chemicals and Membranes

Anion exchange membrane (AEM, denoted as PiperION® Anion Exchange Membrane, 40 microns, Self-Supporting) purchased from FuelCell Store, cation exchange membrane (CEM, denoted as Nafion), and bipolar membrane (BPM, denoted as Fumasep FBM) purchased from FuelCell Store were used in this study. Membranes' characteristics are listed in Table 7. All the chemicals used in this study were of analytical grade, and all the solutions were prepared using Milli-Q water throughout the study.

TABLE 7
Characteristics of commercial membranes used in this study

| | | Ion | | | |
|---|---|---|---|---|---|
| | Thick- | exchange | Ion | Water | |
| Mem- | ness | capacity | conductivity | uptake | Pretreatment |
| brane | (μm) | (meq./g) | (mS/cm) | (wt %) | method |
| CEM | 50 | 0.93 | 145 | 35 | 0.1M NaCl, |
| | | | | | 12 hrs |
| AEM | 40 | 2.35 | 150 | — | 0.1M NaCl, |
| | | | | | 12 hrs |
| BPM | 200-250 | — | — | — | 1M NaCl |

### Experimental Design

In the BMED experiments, the mixed NaOH/Na₂TPA/EG solution and 0.3 M Na₂SO₄ solution were pumped into salt and electrode chambers separately, with a volumetric flow rate of 100 mL/min. During the process, a fixed volume of 200 mL was used for the solutions pumped into the salt, acid, and base chambers. The BMED experiments were performed in a batch mode, where the effluent streams were circulated back to the respective tanks. The electrode solution with a volume of 400 mL was recirculated through anode and cathode chambers separately, with a flow rate of 100 mL/min. The BMED experiment was conducted under the current density of 10 or 20 mA/cm². Samples were collected every two hours to measure the concentration of TPA and the concentrations of ethylene glycol.

### Sample Treatment and Testing Methods.

During BMED experiments, samples with the volume of 1 mL for each one from salt chamber, acid chamber and base chamber were collected every two hours. Totally, the concentration of TPA by using Nuclear Magnetic Resonance (NMR) spectroscopy and the concentrations of ethylene glycol were measured by high-performance liquid chromatography (HPLC).

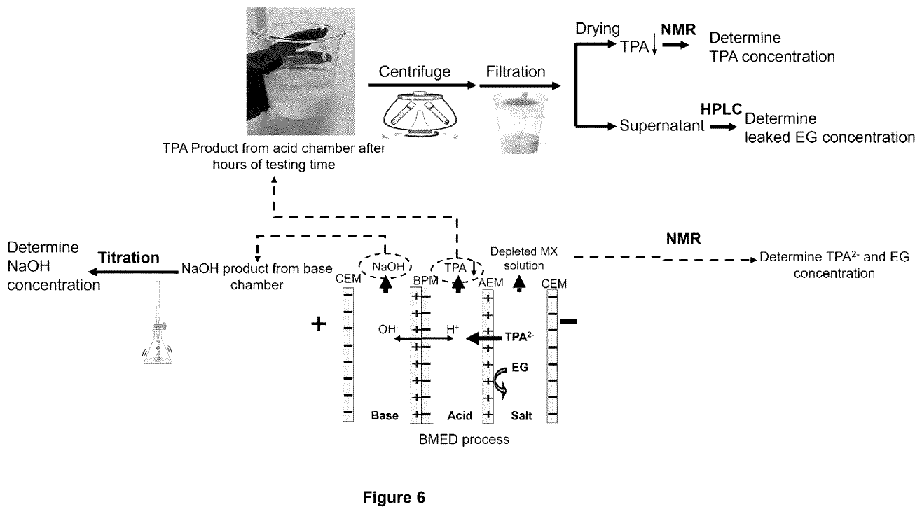

As seen from FIG. 6, the sample from the base chamber was used for detecting NaOH production by BPM. To have more precise determination of NaOH concentration, 1 M H₂SO₄ solution was used for titration. The sample from the salt chamber was used for detecting the concentration variation of EG in salt chamber. 200 μL of a 0.55 M H₂SO₄ was added to a 200 μL sample to adjust pH to 1-3. Then HPLC was used to determine the concentration of ethylene glycol through comparing the peak area of sample to those of standards. The sample from the acid chamber was used for measuring TPA purification by BPM and the amount of ethylene glycol leakage from salt chamber to adjacent acid chamber. Samples were centrifuged to separate TPA precipitate and supernatant. Finally, TPA precipitate and supernatant were separated by filtration. d6-DMSO is used as the NMR solvent to analyse TPA precipitate. The concentration of TPA is calculated by integrating the NMR proton peaks relative to an internal standard (dimethyl sulfone) of a known concentration.

### Calculation

The ratio of EG leakage to acid chamber (Lea_{EG}, %) can be calculated by the following equation:

$$L e {a}_{E G} = \frac{{C}_{A , τ} {V}_{A , τ} - {C}_{A , 0} {V}_{A , 0}}{{C}_{S , 0} {V}_{S , 0}} * 100 \%$$

where C_{A,τ} and C_{A, 0} are the concentrations of ethylene glycol (EG) in acid chamber at time τ and time 0; V_{A,τ} and V_{A, 0} are the volumes of ethylene glycol (EG) in acid chamber at time τ and time 0; C_{S,0} is the concentration of EG in salt chamber at initial time; V_{S,0} is the volume of solution in salt chamber at initial time.

### Bipolar Membrane Electrodialysis Processes for Simulated Depolymerized Solution Treatment

TABLE 8
Initial composition of feed solutions

| | | Concentration | Volume |
|---|---|---|---|
| Solution | Chemical | (mol/L) | (mL) |
| Salt | EG | 0.545 | 200 |
| | Na₂TPA | 0.59 | |
| | NaOH | 0.3 | |
| Acid | NaCl | 0.01 | 200 |
| Base | DI water | NA | 200 |
| Electrode | Na₂SO₄ | 0.3 | 400 |

### Results

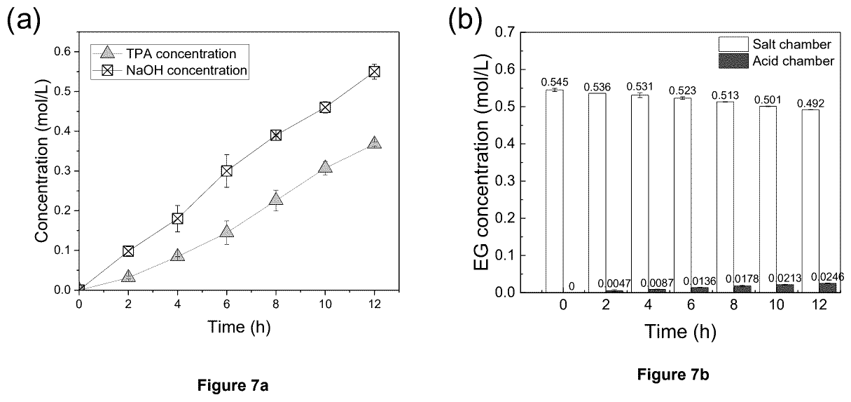

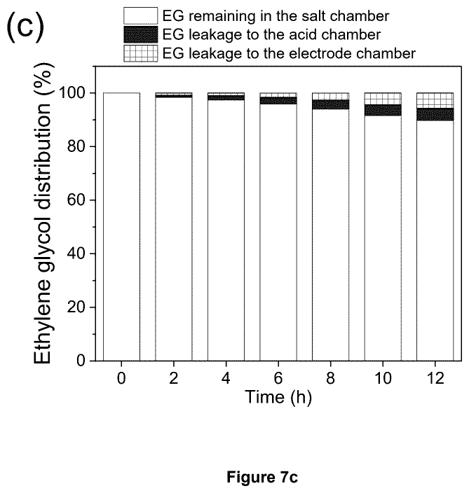

The TPA and NaOH concentration can be shown in FIGS. 7(a) and (b). After 12 h of testing, around 0.37 mol/L TPA could be recovered from 0.6 M Na₂TPA, and around 0.55 mol/L NaOH was produced by BPM.

To investigate the separation effect of TPA²⁻ and ethylene glycol by anion exchange membrane. After 12 hours of BMED testing, approximately 0.22 M TPA²⁻ remained in the feed solution, and the amount of TPA²⁻ could further decrease through prolonging experimental time. The concentration of EG in salt and acid chambers as a function of time is investigated in this study. As can be seen from FIG. 7(b), after 12 hours of testing, only 0.025 mol/L ethylene glycol leaked from the salt chamber to the adjacent acid chamber, amounting to 4.61% EG leakage (FIG. 7(c)). This is because TPA²⁻ is a divalent negative ion, which can transport fast through anion exchange membrane under high current density (strong electric field). EG is a molecule without charge and not influenced by electric field, therefore, AEM will pass very little of it under concentration difference.

### Bipolar Membrane Electrodialysis Processes for Real Depolymerized Solution Treatment

TABLE 9
Initial composition of feed solutions

| | | Concentration | Volume |
|---|---|---|---|
| Solution | Chemical | (mol/L) | (mL) |
| Salt | EG | 0.27 | 200 |
| | Na₂TPA | 0.29 | |
| | NaOH | 0.8 | |
| Acid | NaCl | 0.01 | 200 |
| Base | DI water | NA | 200 |
| Electrode | Na₂SO₄ | 0.3 | 400 |

### Results

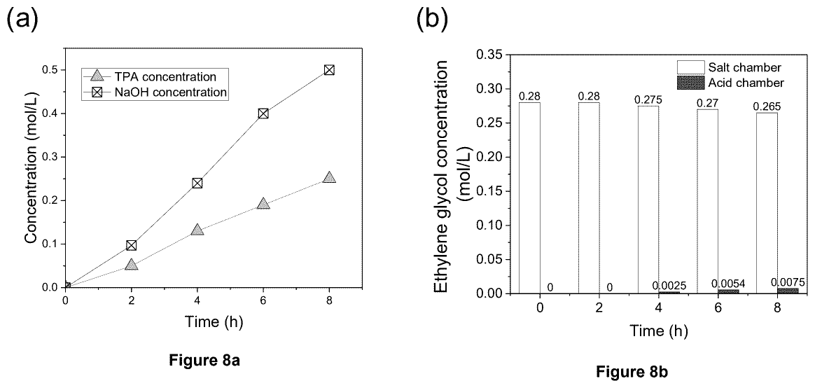

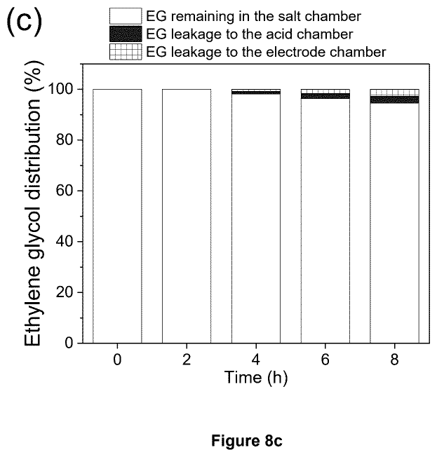

The TPA and NaOH concentration can be shown in FIG. 8(a). After 8 h of testing, only 0.05 M TPA²⁻ remained in the feed solution. around 0.25 mol/L TPA could be recovered from 0.3 M Na₂TPA, and around 0.5 mol/L NaOH was produced by BPM. As can be seen from FIG. 8(b), after 8 hours of testing, there was only 0.0075 mol/L ethylene glycol leaked from salt chamber to adjacent acid chamber, which means around 2.6% ethylene glycol leaked to salt chamber (FIG. 8(c)).

### Specific Energy Consumption

The specific energy consumption for TPA and NaOH production, defined as the energy consumed to produce 1 unit of product, can be calculated using the following equations:

$$S E C = \frac{I * A * {\int }_{0}^{τ} Ud τ}{{C}_{τ} {V}_{c , τ} - {C}_{0} {V}_{c , 0}}$$

where U is the voltage across the ED stack at time τ (V). I is the current density (mA/cm²). C_{τ} is the produced TPA or NaOH concentration (mol/L). C₀ is initial concentration in the respective chamber (mol/L).
Specific Energy Consumption for NaOH and TPA Recovery from Simulated Depolymerized Solution

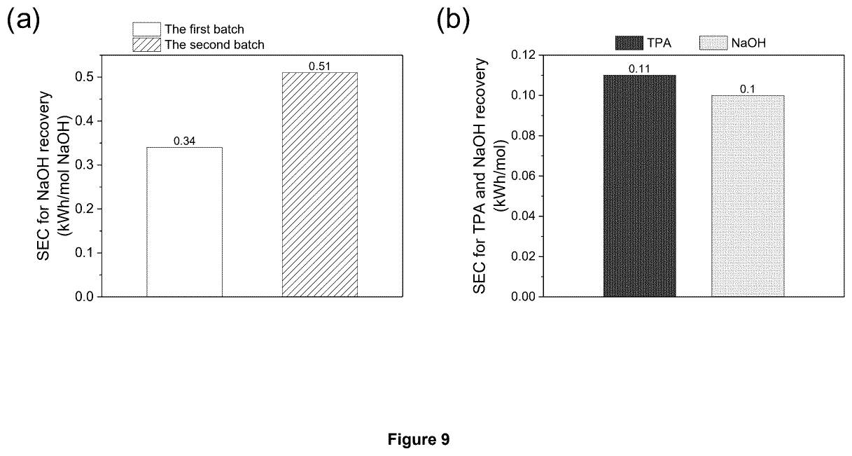

Due to lower initial concentration of NaOH on the second batch, the specific energy consumption for NaOH recovery (0.34 kWh/mol) was much less than that on the first batch (0.51 kWh/mol) (FIG. 9(a)). As seen from FIG. 9(b), the specific energy consumption for TPA and NaOH recovery remained at relatively low values (0.11 kWh/mol for TPA and 0.1 kWh/mol for NaOH).

Specific Energy Consumption for NaOH and TPA Recovery from Real Depolymerized Solution

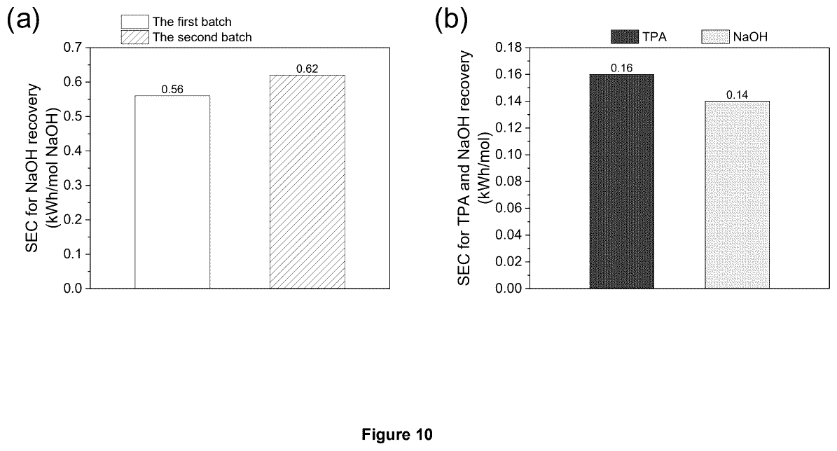

As can be seen from FIG. 10(a), the specific energy consumption for NaOH recovery (0.56 kWh/mol) was slightly less than that on the first batch (0.62 kWh/mol). Besides, from FIG. 10(b), the specific energy consumption for TPA and NaOH recovery remained at relatively low values (0.16 kWh/mol for TPA and 0.14 kWh/mol for NaOH).

### Conclusion

Here we proposed electrodialysis system equipped with only cation exchange membranes for NaOH recovery. It is promising to use BMED process for TPA²⁻ and EG separation and TPA precipitation production. The performance of BMED for TPA and NaOH recovery could be further improved by prolonging the experimental time and developing a more selective and anti-fouling anion exchange membrane.

For treating simulated depolymerized solution, after two batches of running, 0.25 M NaOH and 0.19 M NaOH could be recovered from solution with 0.6 M NaOH, separately. After 12 hours of testing, anion exchange membrane had a very good TPA²⁻/EG separation effect and could recover 0.36 mol/L TPA and 0.55 mol/L NaOH from mixed organic solution. The TPA recovery rate could reach 60% (Table 10).

TABLE 10
Summary of recovered products and energy consumption from simulated depolymerized solution

| | | | Volume ratio | | | Specific |
|---|---|---|---|---|---|---|
| | Initial feed | Final feed | of feed | | | energy |
| | concentration | concentration | solution to | Product | Recovery | consumption |
| Step | (M) | (M) | product | concentration | rate | (kWh/mol) |
| NaOH | Na₂TPA: | Na₂TPA: | 2:1 | NaOH: 0.25M/ | 37% | 0.43/the first |
| recovery | 0.59 | 0.59 | | the first | | batch |
| | EG: 0.59 | EG: 0.545 | | batch | | 0.48/the |
| | NaOH: 0.6 | NaOH: 0.3 | | 0.19M/the | | second |
| | | | | second batch | | batch |
| TPA²⁻/EG | Na₂TPA: | Na₂TPA: | 1:1 | TPA: 0.36M | 61% | 0.11/TPA |
| separation | 0.59 | 0.22 | | NaOH: 0.55M | — | 0.1/NaOH |
| and TPA | EG: 0.545 | EG: 0.49 | | | | |
| production | NaOH: 0.3 | NaOH: 0.1 | | | | |

For treating real depolymerized solution, after two batches of running, 0.28 M NaOH and 0.25 M NaOH could be recovered from solution with 1.4 M NaOH, separately. After 8 hours of BMED testing, 0.2 mol/L TPA and 0.5 mol/L NaOH could be recovered from mixed organic solution. The recovery rate for TPA could reach 83.3% (Table 11).

TABLE 11
Summary of recovered products and energy consumption from real depolymerized solution

| | | | Volume | | | |
|---|---|---|---|---|---|---|
| | | | ratio of | | | Specific |
| | Initial feed | Final feed | feed | | | energy |
| | concentration | concentration | solution to | Product | Recovery | consumption |
| Step | (M) | (M) | product | concentration | rate | (kWh/mol) |
| Module | Na₂TPA: | Na₂TPA: | 1:1 | NaOH: 0.28M/ | 38% | 0.56/the |
| 1: NaOH | 0.3 | 0.3 | | the first | | first batch |
| recovery | EG: 0.29 | EG: 0.27 | | batch | | 0.62/the |
| | NaOH: 1.4 | NaOH: 0.8 | | 0.25M/the | | second |
| | | | | second batch | | batch |
| Module 2: | Na₂TPA: | Na₂TPA: | 1:1 | TPA: 0.25M | 83.3% | 0.11/TPA |
| TPA²⁻/EG | 0.3 | 0.05 | | NaOH: 0.5M | — | 0.1/NaOH |
| separation | EG: 0.27 | EG: 0.26 | | | | |
| and | NaOH: 0.8 | NaOH: 0.8 | | | | |
| TPA | | | | | | |
| production | | | | | | |

### Example 4—PET-Derived Ethylene Glycol Electrolysis

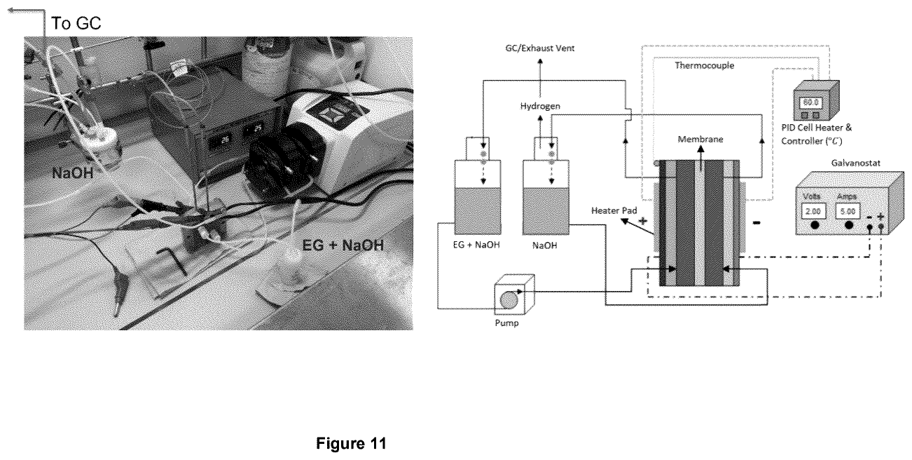

In this step, the EG and NaOH solution separated from step 2 in Example 3 will be fed into a membrane-electrode-assembly electrolyser system coupled with AEM, as shown in FIG. 11.

The reactions happening on the anode and cathode are as follows:
Anode: C₂H₆O₂+4OH⁻→3H₂O+4e⁻+C₂H₄O₃
Cathode: 2H₂O+4e⁻→4OH⁻+2H₂
Overall reaction: C₂H₆O₂+H₂O→C₂H₄O₃+2H₂

### Electrode Preparation

The anodes were prepared by mixing 3 mg Pt/C 60% catalysts (HiSPEC® 9100, Johnson & Matthey), 9 mg Super P® carbon black (Alfa Aesar), 1584 μL ethanol, 4200 μL H₂O, and 216 μL Nafion solution (5% w/w). The mixture was then sonicated for 10 min with probe ultrasonicator to obtain a homogeneous slurry. 3 mL of the slurry was spray coated onto 3*3 cm² carbon fiber paper to achieve a Pt loading of 0.1 mg cm⁻². The cathode used in this reaction was also Pt/C 60% catalyst, prepared with the same spray coating method with a Pt loading of 0.05 mg cm⁻². The loading of the catalysts are a magnitude lower than industrial process, demonstrating the resource efficiency in this process. The prepared electrodes were then placed in a vacuum oven and dried at 60 °C overnight.

### Electrolyte Preparation

The anode electrolyte contains EG and NaOH was prepared by diluting the high concentration reaction solution to achieve 1 M EG+1 M NaOH. The cathode electrolyte contains only 1 M NaOH.

### EG Electrolysis

The electrolysis tests were performed in a modified commercial AEM water electrolyser (Dioxide Materials®, 5 cm² geometric active surface area). AEM (5×5 cm², Fumasep FAA-3-50) were activated in 1 M NaOH solution for at least 24 h before testing. During the measurement, the temperature was maintained at 60 °C. The electrolytes on both sides were circulated by a peristaltic pump to ensure good mass transport. Each electrolysis measurement was done for 1 hour, by holding at the specified current density: 50 mA (10 mA/cm²), 100 mA (20 mA/cm²), 200 mA (40 mA/cm²) and 500 mA (100 mA/cm²). The H₂ produced on the cathode side was analysed in real time by the connected on-line gas chromatography sampling system. After each measurement, the anolyte and catholyte were sampled and analysed by high-performance liquid chromatography to quantify the EG oxidation products. The faradaic efficiency of all products is calculated via:

$$FE \% = \frac{m \times n \times F}{Q} \times 100 \% ,$$

Where m is the detected products in mol, n is the electron transfer required to produce the products (oxalic acid: 8; glycolic acid: 4; glycoxylic acid: 6; formic acid: 3), F is faraday constant 96,485 C mol⁻¹, and Q is the total charge passed.

The power consumption and electricity efficiency in an electrolysis cell per Nm⁻³ of generated H₂ can be calculated from the cell voltage (V_{cell}), expressed as:

$${W}_{e} ( kWh {Nm}^{- 3} ) = {V}_{ceil} \times ( \frac{nF}{t} ) \times ( \frac{1}{24.47} ) = 2.19 \times {V}_{ceil}$$

Where 24.47 L mol⁻¹ is considered as the molar volume of an ideal gas at 25 °C and 1 atm, n is the HER electron transfer (n=2) and F is Faraday constant 96,485 C mol⁻¹, t is the electrolysis time.

Hydrogen production rate of an electrolysis cell at 25 °C can be calculated from:

$${f}_{{H}_{2}} ( \frac{{Nm}^{3}}{h} ) = {η}_{F} \times \frac{I}{nF} \times [ 3600 \times \frac{24.47}{1 \; 000} \frac{\frac{N {m}^{3}}{h}}{\frac{mol}{s}} ]$$

### Results and Discussion:

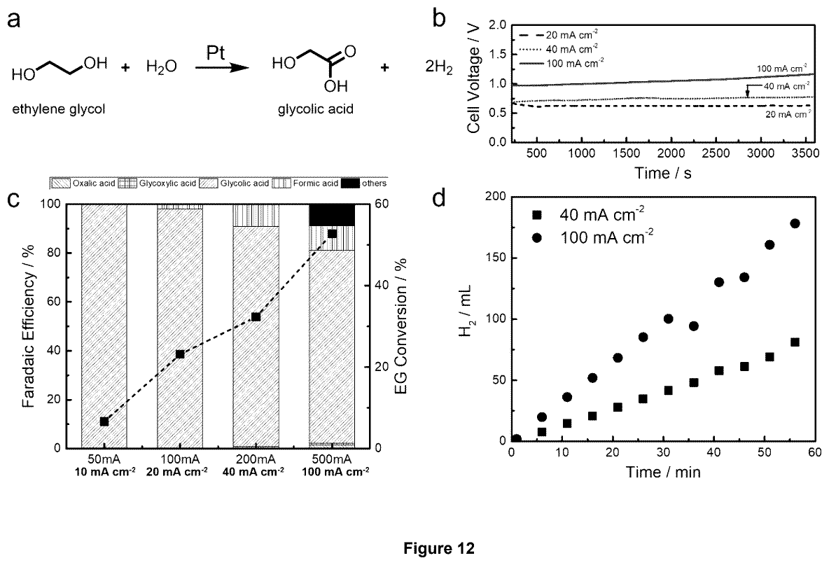

The EG electrolysis results have been summarised in FIG. 12.

The overall reaction process is shown in FIG. 12a. For each mol of EG converted, 1 mol of glycolic acid and 2 mol of H₂ are being generated. FIG. 12b presents the electrolysis profiles when holding at different current densities. It is obvious that with increasing current density, the cell voltage also increases, due to the higher ohmic resistance and over potential for both EG oxidation on the anode and hydrogen evolution on the cathode. At 100 mA cm⁻², the cell voltage is still near 1 V, corresponding to an electricity efficiency Wₑ of 2.2 kWh Nm⁻³ (26.2 kWh kg⁻¹ H₂), which is significantly lower than the current state-of-the-art PEM water electrolyser (51.2 kWh kg⁻¹). The faradaic selectivity towards different products and EG conversion rate at different current densities are shown in FIG. 12c. With increasing current density, the EG conversion rate increases from 6.6% to 52.7%. The majority of the products are glycolic acid, reaching 100% and 99% at 10 and 20 mA cm⁻². Further increasing the current density results in a slight decrease in the selectivity, due to the formic acid and carbonate formation, following the reaction pathway showing below, but still achieving 79% at the end of the electrolysis.

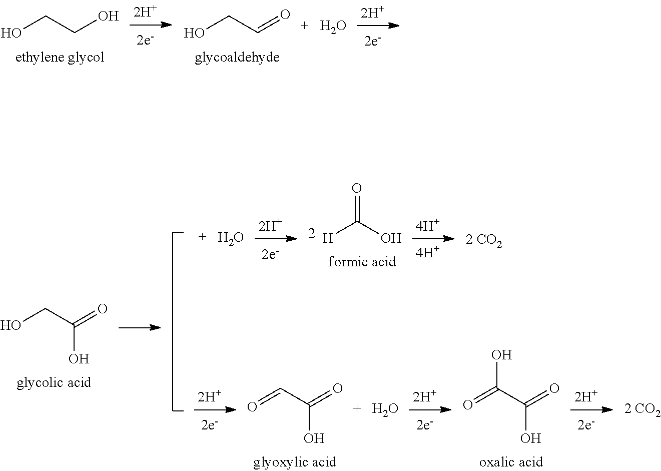

The H₂ produced on the cathode was quantified with online GC. As seen in FIG. 12d, the H₂ stream is steady during the electrolysis period.

### Conclusion:

In conclusion, the 3^{rd} step EG electrolysis has been carried out. The electricity consumption and product distribution have been analysed in detail. With a low loading of commercial Pt/C catalyst on the anode (0.1 mg cm⁻²), we are able to achieve a current density of 100 mA cm⁻², with 79% selectivity towards glycolic acid and an electricity efficiency of 26.2 kWh kg⁻¹ towards H₂ production, nearly 50% lower electricity consumption than the current PEM electrolyser. This process demonstrates that it is possible to produce low-cost green hydrogen by replacing the water electrolysis with PET waste derived EG electrolysis, and the co-production of valuable chemical glycolic acid will further increase the economic impact of the overall process.

### Example 5—EG Electrolysis with Au/C Anode

The electrode and electrolyte preparation follows that of the example with Pt/C in Example 4, except the Au loading on the anode is 0.2 mg cm⁻². The rest of the electrolysis process and product analysis are identical as well.

### Results and Discussion:

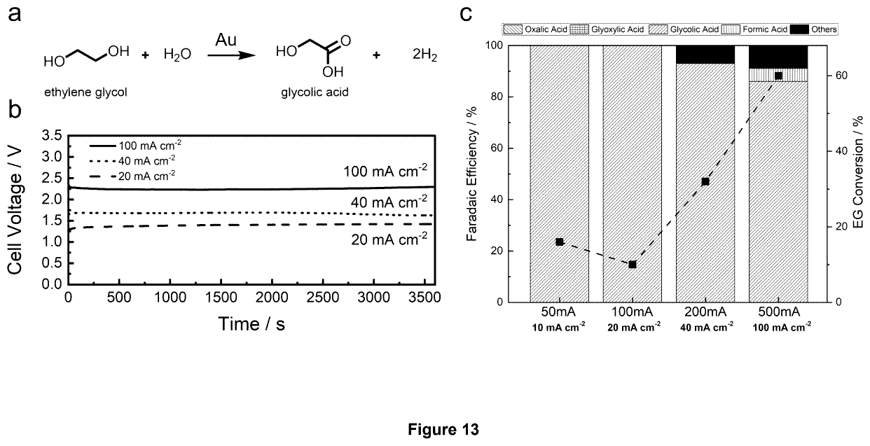

Results are shown in FIG. 13. Compared to using Pt/C as the anode, using Au/C resulted in a higher cell voltage as expected, due to the higher overpotential required for Au to catalyse EG oxidation.

At 100 mA cm⁻², the cell voltage is around 2.3 V, corresponding to an electricity efficiency Wₑ of 5.0 kWh Nm⁻³ (55.79 kWh kg⁻¹ H₂), which is higher than the current state-of-the-art PEM water electrolyser (51.2 kWh kg^{-1r}).

The faradaic selectivity towards different products and EG conversion rate at different current densities are shown in FIG. 13c. With increasing current density, the EG conversion rate increases from 16% to 60%. Glycolic acid is the major product and has a faradaic efficiency of nearly 100% at both 10 and 20 mA cm⁻². Increasing the current density further results in a slight decrease in the selectivity, due to the formation of more oxidised products, such as oxalic acid, as well as formic acid and carbonate. The faradaic efficiency of glycolic acid is still very high at 86% after 1 h at 100 mA cm⁻², compared to the 79% observed when using Pt/C as the anode catalyst. As expected the H₂ production is steady throughout the 1 h experiment.

### Conclusion:

In this work, the EG electrolysis to selectively produce glycolic acid and H₂ has been demonstrated using a low loading of commercial Au/C catalyst on the anode (0.2 mg cm⁻²) coupled with a low loading Pt/C cathode (0.05 mg cm⁻²). At high applied current densities, the faradaic efficiency towards glycolic acid production remains high (>80%).

While preferred embodiments of the present invention have been shown and described herein, it will be apparent to those skilled in the art that such embodiments are provided by way of example only. It will be appreciated that variations to the foregoing embodiments of the invention can be made while still falling within the scope of the invention. Each feature disclosed in this specification, unless stated otherwise, may be replaced by alternative features serving the same, equivalent or similar purpose. Thus, unless stated otherwise, each feature disclosed is one example only of a generic series of equivalent or similar features.

All of the features disclosed in this specification may be combined in any combination, except combinations where at least some of such features and/or steps are mutually exclusive. In particular, the preferred features of the invention are applicable to all aspects of the invention and may be used in any combination. Likewise, features described in non-essential combinations may be used separately (not in combination).

It will be appreciated that many of the features described above, particularly of the preferred embodiments, are inventive in their own right and not just as part of an embodiment of the present invention. Independent protection may be sought for these features in addition to or alternative to any invention presently claimed.

1. A process for degrading polyethylene terephthalate (PET), comprising:
providing PET and mixing the PET with a base;
grinding the PET and base to obtain a ground mixture;
subjecting the ground mixture to aging conditions comprising about 75% or more relative humidity and about 30 °C or more for at least about 24 hours to form an aged ground mixture;
dissolving the aged ground mixture to form a solution comprising terephthalic acid, or a salt thereof, and ethylene glycol;
extracting terephthalic acid from the solution to leave a solution comprising ethylene glycol; and
electrolysing the solution comprising ethylene glycol to produce hydrogen and glycolic acid.

2. The process of claim 1, wherein the base is an Alkali metal salt (for example, sodium hydroxide, lithium hydroxide or potassium hydroxide) or an Alkaline Earth metal salt (for example, calcium hydroxide or magnesium hydroxide), or a mixture thereof.

3. The process of claim 1, wherein the PET and base are mixed with a solvent, optionally wherein the solvent is an aqueous solvent, optionally water.

4. The process of claim 1, wherein the grinding comprises subjecting the mixture to a shearing force.

5. The process of claim 1, wherein the grinding comprises milling or acoustic mixing.

6. The process of claim 1, wherein the grinding comprises milling, optionally wherein the milling is ball milling (e.g. vibratory ball milling or planetary ball milling).

7. The process of claim 1, wherein the amount of base is equal to about 1 molar equivalent or more of the amount of PET, optionally about 1 molar equivalent or more, optionally about 3 molar equivalents.

8. The process of claim 1, wherein the aging conditions comprise:
about 80% or more, about 85% or more, about 90% or more, about 95% or more, about 99% or more, or about 100% relative humidity;
and about 35 °C or more, or about 40 °C or more; and/or
for at least about 36 hours, or for at least about 48 hours.

9. The process of claim 1, wherein the aging is carried out in the presence of water.

10. The process of claim 1, wherein the aged ground mixture is dissolved in an aqueous solvent to form the solution, optionally wherein the solvent is water.

11. The process of claim 1, wherein extracting terephthalic acid from the solution comprising terephthalic acid, or a salt thereof, and ethylene glycol comprises:
a) acidifying the solution, filtering and collecting the terephthalic acid residue; or
b) electrodialysis of the solution to obtain terephthalic acid.

12. The process of claim 1, wherein the electrolysis is carried out in an anion exchange membrane electrolyser.

13. The process of claim 1, wherein:
a) the solution comprising ethylene glycol is used as the anode electrolyte for electrolysis, optionally wherein the base is sodium hydroxide and the process further comprises adjusting the solution comprising ethylene glycol to form an about 1 M ethylene glycol/1 M sodium hydroxide solution prior to electrolysis; and/or
b) a sodium hydroxide solution is used as the cathode electrolyte for electrolysis, optionally an about 1 M sodium hydroxide solution.

14. The process of claim 1, wherein the electrolysis is carried out at a temperature of at least about 40 °C, optionally at least about 50 °C, optionally at least about 60 °C.

15. The process of claim 1, wherein the anode for the electrolysis is a Pt/C, Pd/C or Au/C electrode and/or where the cathode for the electrolysis is a Pt/C, Pd/C or Au/C electrode.

16. The process of claim 1, wherein the electrolysis comprises applying a current density of at least about 5 mA/cm² to the solution comprising ethylene glycol, optionally applying a current density of at least about 10 mA/cm², at least about 20 mA/cm², optionally at least about 30 mA/cm², optionally at least about 40 mA/cm², optionally at least about 50 mA/cm², optionally at least about 75 mA/cm², optionally at least about 100 mA/cm².

17. The process of claim 1, wherein the electrolysis is carried out as a continuous process.

18. A process for degrading polyethylene terephthalate (PET), comprising:
providing PET and mixing the PET with a base and an aqueous solvent, wherein 1 to 5 molar equivalents of each the base and solvent are used compared to the PET;
milling the PET mixture to obtain a ground mixture;
subjecting the ground mixture to aging conditions comprising about 75% or more relative humidity and about 30 °C or more for at least about 24 hours to form an aged ground mixture.

19. The process of claim 18, further comprising dissolving the aged ground mixture to form a solution and extracting terephthalic acid from the solution.

20. The process of claim 18, wherein the aging conditions comprise:
about 80% or more, about 85% or more, about 90% or more, about 95% or more, about 99% or more, or about 100% relative humidity;
and about 35 °C or more, or about 40 °C or more; and/or
for at least about 36 hours, or for at least about 48 hours.

21. The process of claim 1, wherein the aging is carried out in the presence of water.

22. A process for electrolysing ethylene glycol, comprising:
providing a solution comprising ethylene glycol and sodium hydroxide in water as anode electrolyte in an anion exchange membrane electrolyser; and
applying a current density of at least about 5 mA/cm² to the solution to produce hydrogen and glycolic acid;
wherein the solution comprising ethylene glycol and sodium hydroxide in water has a concentration of about 1 M ethylene glycol and about 1M sodium hydroxide;
wherein the cathode electrolyte comprises an about 1 M sodium hydroxide solution;
wherein the anode for the electrolysis is a Pt/C electrode and/or where the cathode for the electrolysis is a Pt/C electrode; and
wherein the electrolysis process is carried out at a temperature of at least about 40 °C.

23. The process of claim 22, wherein:
a) the electrolysis process is carried out at a temperature of at least about 40 °C, optionally at least about 50 °C, optionally at least about 60 °C; and/or
b) a current density of at least about 10 mA/cm² is applied, optionally at least about 20 mA/cm², optionally at least about 30 mA/cm², optionally at least about 40 mA/cm², optionally at least about 50 mA/cm², optionally at least about 75 mA/cm², optionally at least about 100 mA/cm².

24. The process of claim 22, wherein the electrolysis is carried out as a continuous process.

25. Terephthalic acid as prepared by a process according to claim 1.
